(19) United States
(12) Patent Application Publication
(10) Pub. No.: US 2022/0395558 A1
(43) Pub. Date: Dec. 15, 2022

(54) COMPOSITIONS FOR TREATMENT OF ERECTILE DYSFUNCTION, METHODS FOR PREPARING THE SAME AND APPLICATIONS THEREOF

(71) Applicants: Vasanthi PALANIVEL, Bangalore (IN); Shrinivas RANGACHARI, Chennai (IN)

(72) Inventors: Vasanthi PALANIVEL, Bangalore (IN); Shrinivas RANGACHARI, Chennai (IN)

(21) Appl. No.: 17/626,555

(22) PCT Filed: Jul. 13, 2020
(86) PCT No.: PCT/IB2020/056574

§ 371 Date: Jan. 12, 2022

(30) Foreign Application Priority Data
Jul. 12, 2019 (IN) 201941028136

(51) Int. Cl. A61K 38/18; A61K 35/19; A61K 35/15

(52) U.S. Cl. CPC A61K 38/1858; A61K 35/19; A61K 35/15; A61K 38/185; A61K 38/1825

(57) ABSTRACT

The present disclosure generally relates to the field of infertility, and in particular male infertility. Accordingly, the present disclosure provides for compositions and methods for managing male infertility, caused by erectile dysfunction. More particularly, the present disclosure provides a therapeutic composition comprising a platelet rich plasma (PRP) or a growth factor concentrate derived therefrom and a thermoresponsive polymer. The present disclosure also relates to the compositions of PRP and the concentrate themselves. Consequently, methods to obtain the said compositions, along with therapeutic applications for treatment of erectile dysfunction are also provided.

### TECHNICAL FIELD

The present disclosure relates to the compositions, kits, and methods for treating male infertility. In particular, the present disclosure relates to the compositions, kits, and methods for treating erectile dysfunction. The present disclosure relates to methods for preparing compositions for treating erectile dysfunction.

### BACKGROUND OF THE DISCLOSURE

Erectile dysfunction (ED) is defined as the inability to attain or maintain a penile erection satisfactory for sexual intercourse. ED is a prevalent health problem that seriously impacts the quality of life of men and their partners. It is estimated that approximately 50% of men between the ages of 40 and 70 years have some degree of ED. The majority of ED patients are treated with phosphodiesterase type-5 inhibitors (PDE5i), such as sildenafil, vardenafil, tadalafil, and avanafil. However, PDE5 is can cause a variety of side effects that make them unsuitable for some patients, and they are contraindicated in patients who also take nitrates because of the danger of synergistic hypotensive effects. Several other management options exist for ED, including lifestyle modifications and pharmacotherapeutic strategies such as intraurethral alprostadil, intracorporal injection therapy, vacuum erection devices, and surgery, including penile revascularization and penile prosthesis implantation. Despite the efficacy of these modalities, limitations to their use exist, such as intolerance to side effects, cost limitations, and unsatisfactory outcomes.

With the exceptions of lifestyle modification and revascularization procedures, these methods merely treat the manifestations of ED, offering only symptomatic relief. The pressing need to develop a curative treatment for ED has stimulated interest in utilizing stem cell (SC) therapies for treating ED patients. With the exceptions of lifestyle modification and revascularization procedures, these methods merely treat the manifestations of ED, offering symptomatic relief rather than a cure for the underlying disease process. The pressing need to develop a curative treatment for ED has stimulated interest in utilizing stem-cell therapy in ED patients [Soebadi et al., 2016]. Various groups worldwide are currently involved in investigating how cell-based therapy, specifically SCs, might be of use in reversing different pathophysiological processes in the establishment of ED to halt or reverse the development of this prevalent sexual dysfunction. The regenerative effects of SC are likely achieved by secretion of various growth factors into the blood stream and/or migration of these factors to major pelvic ganglia in addition to cell contact, paracrine signalling system and cellular differentiation.

The first reported clinical trial of SC therapy in diabetic men with ED showed a reasonable increase in penile rigidity after a single intracavernous injection of umbilical cord blood SC. While penile rigidity was maintained for more than 6 months, the erection was not hard for sexual penetration, suggesting that the amount and a single administration of SC were likely insufficient for adequate penile rigidity. In a different study on the use of SC in men with ED following radical prostatectomy, Yiou et al., showed that intracavernous injection of bone marrow mononuclear cells appeared to be safe and improved the erectile function for a period of 6 months.

The intracavernous injection of SC to treat ED appears straightforward and logical with proposed regenerative effect is achieved by either secreting growth factors locally via a paracrine mechanism or by migration to the major pelvic ganglia, to promote the propagation and differentiation of resident progenitor cells and encourage the recovery of injured tissue via the production of antiapoptotic and proangiogenic factors, rather than transdifferentiation into different cell types. Adult SC has the advantage of avoiding the ethical issues of ESC and in addition, published literature shows a very low probability of malignant transformation and tumour formation. While these studies are mainly conducted in a preclinical setting and pilot clinical studies, clinical trials are starting to emerge based on positive preclinical results, and the outcome of these studies might change the approach towards ED.

In the SC therapies, SCs are injected into the corpus cavernosum of the penis (intracavernous injection). The regenerative effects of the SC therapy are likely achieved by secretion of various growth factors into the blood stream and/or migration of these factors to major pelvic ganglia in addition to cell contact, paracrine signalling system and cellular differentiation.

Tissue engineering traditionally stimulates cells using a single bioactive agent with key regenerative functions. For example, use of G-CSF for endometrial regeneration. In contrast, natural tissue regeneration relies on a cocktail of signalling molecules and growth factors. During natural wound healing, activated platelets concentrate in the wound area and secrete a plethora of factors that play an instrumental role in not only coordinating wound healing but also in establishing normal tissue architecture and efficient tissue remodelling.

Using a single growth factor to steer tissue regeneration represents an oversimplified and inefficient stimulus. This is generally overcome by providing supraphysiological quantities of the growth factors. As against other specialties, in ART/IVF procedures, every event is time bound and to avoid cycle cancellation, preparation of endometrium in the current cycle is very crucial which is difficult by single bioactive agent like G-CSF.

Platelet rich plasma is another option used in multiple specialties for promoting tissue regeneration. PRP injections have also recently been marketed as a form of autologous cell therapy under the banner of regenerative medicine for treating erectile dysfunction (ED).

While some studies with PRP have shown improved erectile function, there are still some drawbacks associated with current methods. For example, during or after intracavernous or intracorporeal injection, the solution containing stem cells can leak out of the penis thereby decreasing the efficacy of the treatment. Moreover, platelet derived growth factors and other regenerative proteins secreted by stem cells are released at once or over a relatively short duration of time, thereby providing a shorter duration of action. Thus, there is a need to develop therapeutic compositions that are easy to administer, do not leak out, and provide a sustained release of growth factors for treatment of ED. The present disclosure attempts to solve this problem.

### SUMMARY OF THE DISCLOSURE

In some embodiments, the present disclosure relates to a therapeutic composition comprising a platelet rich plasma (PRP) or a growth factor concentrate derived therefrom and a thermoresponsive polymer.

In some embodiments, the present disclosure relates to a therapeutic composition comprising a platelet rich plasma (PRP) or a growth factor concentrate derived therefrom, peripheral blood stem cells (PBSCs), and a thermoresponsive polymer.

In some embodiments, the present disclosure relates to a method for preparing the therapeutic composition as recited above, comprising mixing the PRP or the growth factor concentrate derived therefrom, optionally mixing PBSCs, with the thermoresponsive polymer to obtain the composition.

In some embodiments, the present disclosure relates to use of a thermoresponsive polymer for preparing a medicament for improving fertility.

In some embodiments, the present disclosure relates to a therapeutic composition comprising a platelet rich plasma (PRP) or a growth factor concentrate derived therefrom and a thermoresponsive polymer, for use in treating erectile dysfunction in a subject in need thereof.

In some embodiments, the present disclosure relates to a method for treating erectile dysfunction in a subject in need thereof comprising, administering to the subject the therapeutic composition of the present disclosure.

In some embodiments, the present disclosure relates to a kit for preparing the therapeutic compositions herein, comprising:
a. a RBC activating agent selected from a group comprising: heparin, collagen, a calcium salt, hyaluronic acid, polygeline, thrombin, gelatin, EDTA, sodium citrate, starch, and a combination thereof;
b. a thermoresponsive polymer; and
c. an instruction manual.

In some embodiments, the present disclosure relates to a platelet rich plasma (PRP), wherein:
a. the PRP comprises a platelet count that is about 10 to 20-fold greater than starting whole blood sample from same subject, or
b. a red blood cell (RBC) count that is about 60 to 90-fold lower than starting whole blood sample from same subject, or
c. a white blood cell (WBC) count that is about 10 to 99-fold lower than starting whole blood sample from same subject.

In some embodiments, the present disclosure relates to a platelet-derived growth factor concentrate obtained from the PRP as recited above.

### BRIEF DESCRIPTION OF THE ACCOMPANYING DRAWINGS

FIG. 1 represents chemical formula (A) and representation of volume phase transition (B) between coil (left) and globular (right) hydrogel conformations of a NIPAM based polymer.

FIG. 2 represents (A) the swollen PNIPAAm hydro-sol in aqueous solution below critical temperature (Tc) of 32° C. and (B) the shrunken dehydrated PNIPAAm hydrogel above critical temperature (Tc) of 32° C.

FIG. 3 represents schematic for preparing the composition of the present disclosure and the subsequent administration into penis.

FIG. 4 represents impact of RBC aggregators in the PRP/GFC protocol.

FIGS. 5a-5f represent the growth factor profile of GFC.

FIG. 6 represents the in vitro growth factor release kinetics for comparing the composition of the present disclosure with a preparation devoid of the thermoresponsive polymer.

FIG. 7, panels A-H, show the images of various stages of whole blood processing for preparing the PRP and the GFC of the present disclosure. Panel A shows whole blood drawn from a patient and collected into into acid citrate dextrose (ACD-A) solution gel tube/K2 EDTA tube. Panel B shows settling of RBCs upon incubation of the whole blood for 45 minutes with a buffer comprising one or more RBC aggregating agents. Panel C shows the whole blood after first centrifugation at 600 rpm for 2 minutes—the bottom layer contains RBCs and WBCs and the supernatant contains platelets-containing plasma. Panel D shows the supernatant containing platelets-containing plasma transferred to another centrifugation tube. Panel E shows the platelet pellet obtained after the second centrifugation step at 3000 rpm for 10 minutes. Panel F shows the gel-like consistency of PRP during the platelet-activation stage. Panels G and H show separation of platelets in the form of a clot-like structure from the supernatant containing the growth factor concentrate.

FIG. 8 depicts a comparison of the RBC and WBC count between the GFC of the present disclosure and the starting whole blood.

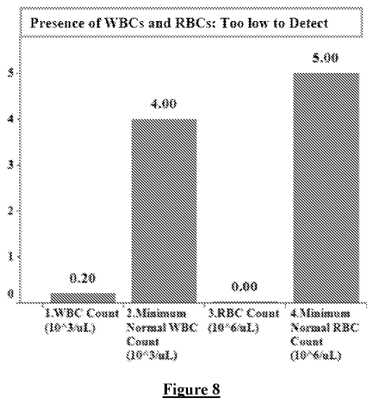

### DETAILED DESCRIPTION OF THE DISCLOSURE

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” or “containing” or “has” or “having” wherever used, will be understood to imply the inclusion of a stated element, integer or step, or group of elements, integers or steps, but not the exclusion of any other element, integer or step, or group of elements, integers or steps.

Reference throughout this specification to “one embodiment” or “some embodiments” means that a particular feature, structure or characteristic described in connection with the embodiment may be included in at least one embodiment of the present disclosure. Thus, the appearances of the phrases “in one embodiment” or “in some embodiments” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

The present disclosure provides therapeutic compositions, methods for preparing said compositions, methods for using said compositions in treating ED, and kits for preparing said therapeutic compositions.

In some embodiments, the present disclosure provides therapeutic compositions comprising platelet-rich plasma (PRP) or a growth factor concentrate derived therefrom and a stimulus responsive polymer such as a thermoresponsive polymer. In some embodiments, the therapeutic composition further comprises peripheral blood stem cells (PBSCs). In some embodiments, also provided herein are therapeutic compositions comprising platelet-rich plasma (PRP) or a growth factor concentrate derived therefrom and PBSCs.

The PRP employed in the compositions of the present invention could be conventional, or specifically prepared as per the protocol provided in the present disclosure. Similarly, the growth factor concentrate derived from PRP can also be prepared from conventional PRP or PRP prepared as per the protocol provided in the present disclosure.

The platelet rich plasma (PRP) or the growth factor concentrate derived therefrom provides for enhanced treatment of ED and the inclusion of a stimulus responsive polymer, particularly a thermoresponsive polymer, helps in greater retention of the composition at the site of the administration and provides a sustained release of growth factors and other therapeutic agents from the composition. Thus, the present disclosure provides for technically advanced compositions that help men suffering from ED recover erectile function at levels much higher than those observed with other currently known technologies, including use of conventional PRP without such a thermosensitive polymer.

Before describing the compositions of the present disclosure, the corresponding methods and the applications thereof in greater detail, it is important to take note of the common terms and phrases that are employed throughout the instant disclosure for better understanding of the technology provided herein.

Throughout the present disclosure, the term “platelet rich plasma (PRP)” is used to mean conventional PRP or the PRP prepared specifically by the method of the present disclosure. Thus, unless otherwise specifically stated, the general use of the term “platelet rich plasma” or “PRP” throughout the disclosure is understood to interchangeably mean conventional PRP or the PRP prepared by the method of the present disclosure. The PRP prepared by the method of the present disclosure is also referred to herein as the “PRP prepared by the present disclosure” or the “PRP of the present disclosure”. While the method specifically employed to prepare PRP in the present disclosure is explained in greater detail below, the conventional PRP is any PRP known in the art prepared by previously known methods and technologies, including the buffy coat method. A person skilled in the art is therefore able to refer to the literature and common general knowledge to prepare the conventional PRP quite easily. An example of methods for preparing the conventional PRP is summarized in a review article entitled “Principles and Methods of Preparation of Platelet-Rich Plasma: A Review and Author's Perspective”, J Cutan Aesthet Surg. 2014 October-December; 7(4): 189-197.

Throughout the present disclosure, the terms “growth factor concentrate” or “platelet-derived growth factor concentrate” or “platelet growth factor concentrate” or “GFC” are used interchangeably and refer to a substantially cell-free supernatant comprising a milieu of growth factors, cytokines, and other proteins obtained from lysis of activated platelets from the platelet rich plasma (PRP). As mentioned above, this PRP could be either a conventional PRP or PRP prepared by the present disclosure. The growth factor concentrate of the present disclosure is substantially free of cells as upon obtaining of the PRP, the activated platelets are lysed for the said preparation of the growth factor concentrate. The ruptured platelets are then allowed to settle down, and the substantially cell-free supernatant is collected. Preferably, the growth factor concentrate is prepared from the PRP of the present disclosure, which is characterized by high platelet count and very low RBC and WBC count compared to the conventional PRP. As the PRP of the present disclosure has high platelet count and very low levels of RBC and WBC contamination compared to conventional PRP, the growth factor concentrate prepared from the PRP prepared by the present disclosure also has improved characteristics than conventional PRP or growth factor concentrates prepared from conventional PRP.

Throughout the present disclosure, the term “stimulus responsive polymer” is used to mean a polymer that is sensitive to or responds to one or more stimuli, which include thermal stimuli, optical stimuli, mechanical stimuli, pH stimuli, chemical stimuli, environmental stimuli or biological stimuli. Preferably, the stimulus responsive polymers employed in the present disclosure are polymers that are sensitive or responsive to thermal stimuli or temperature change. Accordingly, the stimulus responsive polymer is preferably used to mean a thermoresponsive polymer in the context of the present disclosure. These polymers are temperature-responsive polymers that exhibit a drastic and discontinuous change of their physical properties with change in temperature. For example, these polymers could be in liquid form at certain temperatures, and have the ability of quickly converting into a gel form at increased temperatures.

Throughout the present disclosure, since each of the compositions provide for a therapeutic effect in treatment of male infertility caused due to ED, the term “composition” is also meant to be understood as “therapeutic composition” and the two are used interchangeably herein.

### Therapeutic Compositions

Accordingly, to reiterate, in some embodiments, the present disclosure relates to compositions having a PRP or a growth factor concentrate derived therefrom along with a stimulus responsive polymer, preferably a thermoresponsive polymer. In some embodiments, the compositions further comprise PBSCs. In some embodiments, the present disclosure also provides compositions having a PRP or a growth factor concentrate derived therefrom along with PBSCs. The compositions are used for treatment of men suffering from infertility, caused due to ED. As mentioned, the PRP employed in the compositions of the present disclosure could be a conventional PRP or a PRP prepared by the present disclosure. Accordingly, the growth factor concentrates employed in the compositions herein, are also in turn obtained from the corresponding PRP.

### PRP

While the compositions of the present disclosure could comprise of conventional PRP as well as PRP prepared by the present disclosure, in some embodiments, the PRP is preferably the PRP prepared by the present disclosure. The PRP prepared by the present disclosure is enriched in platelets and comprises very low count of red blood cells (RBCs) and white blood cells (WBCs) compared to PRPs known in the art (conventional PRPs). In some embodiments, the PRP of the present disclosure comprises about 10 to 20-fold higher platelet count, 60 to 90-fold lower RBC count, and/or 10 to 99-fold lower WBC count, including values and ranges therebetween, compared to the starting whole blood sample obtained from the same subject.

The PRP of the present disclosure is preferably autologous. However, allogenic PRP and use of allogenic PRP is also contemplated. In some embodiments, the PRP is prepared from venous blood. In some embodiments, the PRP is prepared from cord blood or bone marrow.

In some embodiments, the number of platelets, RBCs, and/or WBCs present in the PRP of the present disclosure are characterized in terms of fold increase or fold decrease compared to the starting whole blood sample or conventional PRPs as the number of platelets, RBCs, and WBCs vary from a subject to subject or even for the same subject over the period of time; accordingly, a fold increase/enrichment (for platelets) and/or a fold decrease/reduction (for RBCs/WBCs) effectively characterize or distinguish the PRP of the present disclosure over starting whole blood sample and/or conventional PRPs.

In some embodiments, the PRP of the present disclosure comprises about 10, 11, 12, 13, 14, 15, 16, 17, 18, 19, or 20-fold more platelets, including values and ranges therebetween, compared to the starting whole blood sample from which the PRP is prepared. In some embodiments, the PRP of the present disclosure comprises about 10 to 20-fold, 10 to 18-fold, 10 to 15-fold, 12 to 20-fold, 12 to 18-fold, 12 to 15-fold, 10 to 12-fold, 10 to 13-fold, 11 to 14-fold, 12 to 14-fold, 12 to 15-fold, 13 to 18-fold, or 15 to 20-fold more platelets, including values and ranges therebetween, compared to the starting whole blood sample. In an exemplary embodiment, if the starting whole blood sample of a subject comprises about 150×10³ platelets per microliter, the PRP prepared according to the present disclosure can comprise about 2040 platelets per microliter, which is about 13.6-fold greater than the starting whole blood sample. In another exemplary embodiment, for a whole blood sample of a subject comprising about 230×10³ platelets per microliter, the PRP of the present disclosure comprises platelets in the range of about 2300 to 4600×10³ per microliter, which is about 10 to 20-fold greater than the starting whole blood sample.

In some embodiments, the platelet count of the PRP of the present disclosure is about 1.2 to 2.5-fold, including values and ranges therebetween, greater than the platelet count of the conventional PRP. In some embodiments, the platelet count of the PRP of the present disclosure is about 1.2, 1.3, 1.4, 1.5, 1.6, 1.7, 1.8, 1.9, 2, 2.1, 2.2, 2.3, 2.4, or 2.5-fold, including values and ranges therebetween, greater than the platelet count of the conventional PRP. In some embodiments, the platelet count of the PRP of the present disclosure is about 1.2 to 2.2-fold, about 1.2 to 2-fold, about 1.2 to 1.8-fold, about 1.2 to 1.6-fold, about 1.5 to 2.5-fold, 1.5 to 2.2-fold, about 1.5 to 2-fold, including values and ranges therebetween, greater than the platelet count of the conventional PRP.

In some embodiments, the RBC count of the PRP of the present disclosure is about 60 to 90-fold lower, including values and ranges therebetween, compared to the starting whole blood sample. In some embodiments, the RBC count of the PRP of the present disclosure is about 60 to 90 fold, about 60 to 85-fold, about 60 to 80-fold, about 60 to 75-fold, about 60 to 70-fold, about 65 to 90-fold, about 65 to 85-fold, about 65 to 80-fold, about 65 to 70-fold, about 65 to 75-fold, about 70 to 80-fold, about 75 to 80-fold, about 70 to 90-fold lower, including values and ranges therebetween, compared to the starting whole blood sample. In some embodiments, the RBC count of the PRP of the present disclosure is about 60, 65, 70, 75, 80, 85, or 90-fold lower, including values and ranges therebetween, compared to the starting whole blood sample. In an exemplary embodiment, if the starting whole blood sample of a subject comprises about 4.7×10⁶ RBCs per microliter, the PRP prepared according to the present disclosure comprises about 0.06×10⁶ RBCs per microliter, which is about 78.3-fold reduction in RBCs than the starting whole blood sample. In another exemplary embodiment, for a whole blood sample of a subject comprising about 5.5×10⁶ RBCs per microliter, the PRP of the present disclosure comprises RBCs in the range of about 0.09 to 0.061×10⁶ per microliter, which is about 60 to 90-fold lower than the starting whole blood sample.

In some embodiments, the RBC count of the PRP of the present disclosure is about 145 to 155-fold, including values and ranges therebetween, reduced compared to the RBC count of the conventional PRP prepared using a single spin method. In some embodiments, the RBC count of the PRP of the present disclosure is about 145 to 150-fold, including values and ranges therebetween, lower than that of the conventional PRP prepared using the single spin method. In some embodiments, the RBC count of the PRP of the present disclosure is about 15 to 25-fold, or about 15 to 20-fold, or about 18 to 22-fold, including values and ranges therebetween, lower than the RBC count of the conventional PRP prepared using a double spin method.

In some embodiments, the WBC count of the PRP of the present disclosure is about 10 to 99-fold lower, including values and ranges therebetween, compared to the starting whole blood sample. In some embodiments, the WBC count of the PRP of the present disclosure is about 10 to 99-fold, about 10 to 80-fold, about 10 to 70-fold, about 10 to 60-fold, about 10 to 50-fold, about 10 to 40-fold, about 10 to 30-fold, about 10 to 25-fold, about 10 to 20-fold, about 15 to 30-fold, about 20 to 30-fold, or about 22 to 28-fold lower, including values and ranges therebetween, compared to the starting whole blood sample. In some embodiments, the WBC count of the PRP of the present disclosure is about 10, 11, 12, 13, 14, 15, 16, 17, 18, 19, 20, 21, 22, 23, 24, 25, 26, 27, 28, 29, 30, 40, 50, 60, 75, 80, or 90-fold lower, including values and ranges therebetween, compared to the starting whole blood sample. In an exemplary embodiment, if the starting whole blood sample of a subject comprises about 4.5×10³ WBCs per microliter, the PRP prepared according to the present disclosure comprises about 0.19×10³ WBCs per microliter, which is about 23.6-fold reduction in WBCs than the starting whole blood sample. In another exemplary embodiment, for a whole blood sample of a subject comprising about 6.5×10³ WBCs per microliter, the PRP of the present disclosure comprises WBCs in the range of about 0.65 to 0.07×10³ per microliter, which is about 10 to 90-fold lower than the starting whole blood sample.

In some embodiments, the WBC count of the PRP of the present disclosure is about 50 to 70-fold, about 55 to 65 fold, or about 55 to 70-fold, including values and ranges therebetween, reduced compared to the WBC count of the conventional PRP. In some embodiments, the WBC count of the PRP of the present disclosure is about 60, 61, 62, 63, 64, 65, 66, 67, 68, 69, or 70-fold, or about 60 to 70-fold, including values and ranges therebetween, lower than that of the conventional PRP prepared using the single spin method. In some embodiments, the WBC count of the PRP of the present disclosure is about 55, 56, 57, 58, 59, 60, 61, 62, 63, 64, or 65-fold, or about 55 to 65-fold, including values and ranges therebetween, lower than the WBC count of the conventional PRP prepared using a double spin method.

In some embodiments, the PRP of the present disclosure comprises about 1500-6750×10³ platelets per microliter, including values and ranges therebetween; about 0.05-0.1×10⁶ RBCs per microliter, including values and ranges therebetween; and/or about 0.1-0.45×10³ WBCs per microliter, including values and ranges therebetween.

In some embodiments, even if the platelet count of the PRP of the present disclosure is marginally higher or closer or may overlap with the platelet count of the conventional PRP; the RBC and/or the WBC count of the PRP of the present disclosure are substantially lower than those of the conventional PRP. In other words, the present PRP has substantially more fold reduction in the RBC count and/or the WBC count than the conventional PRP.

The present disclosure contemplates that the PRP can have any one of the cell counts, fold increase, and fold decrease features described herein, or a combination thereof. For example, in one embodiment, the PRP comprises a platelet count that is about 10 to 20-fold greater, including values and ranges therebetween, than starting whole blood sample. In another exemplary embodiment, the PRP comprises a platelet count that is about 10 to 20-fold greater, including values and ranges therebetween, and a RBC count that is 60 to 90-fold lower, including values and ranges therebetween, than starting whole blood sample. In another exemplary embodiment, the PRP comprises a platelet count that is about 10 to 20-fold greater, including values and ranges therebetween, than starting whole blood sample and a WBC count that is 10 to 99-fold lower, including values and ranges therebetween, than starting whole blood sample from same subject. In another embodiment, the PRP comprises a platelet count that is about 10 to 20-fold greater, including values and ranges therebetween; a RBC count that is 60 to 90-fold lower, including values and ranges therebetween; and a WBC count that is 10 to 99-fold lower, including values and ranges therebetween, than starting whole blood sample from same subject.

In an exemplary embodiment, a platelet pellet obtained from 10 ml of whole blood sample drawn from a patient is resuspended in 1 ml of platelet poor plasma to provide 1 ml of PRP which can be employed in preparing the compositions of the present disclosure or which is further processed to prepare growth factor concentrate (GFC) as described herein. Throughout this disclosure, if the concentration of PRP is expressed in terms of percentages, it refer to the volume of PRP added to the composition—e.g., 30% PRP means 300 μl of PRP is added to make 1 ml of the composition or 3 ml of PRP is added to make 10 ml of the composition.

In some embodiments, the present disclosure provides compositions comprising PRP of the present disclosure and a pharmaceutically acceptable excipient.

### GFC

It is known in the art that platelets serve as a reservoir of growth factors, cytokines, and other proteins. These growth factors, cytokines, and several other proteins are contained in the alpha-granules of platelets and are released upon activation of platelets. Exemplary growth factors present in the alpha-granules of platelets include, but are not limited to, platelet-derived growth factor (PDGF), transforming growth factor (TGF), platelet-derived angiogenesis factor (PDAF), vascular endothelial growth factor (VEGF), epidermal growth factor (EGF), insulin-like growth factor (IGF), basic fibroblast growth factor (bFGF), stromal cell derived factor 1 (SDF-1), and hepatocyte growth factor (HGF). Accordingly, in some embodiments, compositions herein comprise the growth factor concentrate obtained from PRP along with the thermoresponsive polymer.

The present disclosure therefore provides a therapeutic composition having the GFC the thermoresponsive polymer, wherein the growth factor concentrate comprises growth factor(s) selected from a group comprising VEGF, EGF, bFGF, IGF-1, PDGF-BB and TGF-b1 or any combination thereof.

As mentioned, the GFC employed in the present disclosure is prepared from the PRP, which could be conventional PRP or the PRP prepared by the present disclosure. The GFC is prepared by subjecting the activated platelets in the PRP to one or more platelet-activating treatments. These are described in further details in the later paragraphs of the present disclosure.

As the term suggests, the GFC is a concentrated form of growth factors that are originally present in the platelets. Upon platelet-activating treatment, the activated platelets release the said growth factors in the plasma. Accordingly, the concentration of the growth factors in the GFC is about 4 to 10-fold, about 4 to 8-fold, about 5 to 10-fold, about 5 to 8-fold, about 6 to 10-fold, or about 6 to 8-fold, including values and ranges therebetween, higher than that of the starting whole blood sample.

As was the case with PRP, while the GFC can be prepared from conventional PRP, in some embodiments, it is preferred that the GFC is obtained from the PRP prepared by the present disclosure. Exemplary levels of certain growth factors in the growth factor concentrate of the present disclosure vs those obtained from conventional PRP are shown in Table 1 below:

TABLE 1
Exemplary concentration of growth factors in freshly-prepared GFC

| | Concentration | |
|---|---|---|
| | range in the | |
| | freshly-prepared | Concentration |
| Growth | GFC derived from | range in the freshly- |
| Factor | conventional PRP | prepared GFC |
| VEGF | 500-800 pg/mL | 500-1300 pg/mL |
| EGF | 100-200 pg/mL | 100-2000 pg/mL |
| bFGF | 25-75 pg/mL | 25-500 pg/mL |
| IGF-1 | 70-130 ng/mL | 500-1000 ng/mL |
| PDGF-BB | 20-85 ng/mL | 20-500 ng/mL |
| TGF-β1 | 250-350 ng/mL | 100-2000 ng/mL |

In some embodiments, in addition to growth factors from autologous blood, therapeutic compositions are further fortified with exogenously added growth factors to provide a concentration of growth factors that is about 4 to 10 times higher than the baseline concentration of corresponding growth factors in starting whole blood. Accordingly, in some embodiments, in the therapeutic compositions, concentration of the VEGF ranges from about 500 to 3000 pg/mL, concentration of the EGF ranges from about 100 to 3000 pg/mL, concentration of the bFGF ranges from about 25 to 3000 pg/mL, concentration of the IGF-1 ranges from about 500 to 3000 ng/mL, concentration of the PDGF-BB ranges from about 20 to 3000 ng/mL, and concentration of the TGF-β1 ranges from about 100 to 3000 ng/mL.

In an exemplary embodiment, a platelet pellet obtained from 10 ml of whole blood sample drawn from a patient is resuspended in 1 ml of platelet poor plasma to provide 1 ml of PRP which is further processed to obtain 500 μl of the growth factor concentrate (GFC) as described herein. Throughout this disclosure, if the concentration of GFC is expressed in terms of percentages, it refers to the volume of GFC added to the composition—e.g., 30% GFC means 300 μl of GFC is added to make 1 ml of the composition or 3 ml of GFC is added to make 10 ml of the composition.

Once obtained, the platelet-derived growth factor concentrate (GFC) can be put to application instantly or may be subjected to storage for subsequent use. In a non-limiting embodiment, the GFC is stored in airtight vials. Storage without diminished quality is feasible for a period of about 6 months, at a storage temperature ranging from about minus 196° C. to 4° C.

In some embodiments, the present disclosure provides compositions comprising the GFC and a pharmaceutically acceptable excipient.

### PBSCs

Apart from the PRP or the GFC and the thermoresponsive polymer, in some embodiments, the compositions of the present disclosure also comprise peripheral blood stem cells (PBSCs) or endothelial progenitor cells. These PBSCs are a direct result of Endogenous Stem Cell Mobilisation (ESCM) done prior to preparing the composition. Combining the compositions with PBSCs proves to be effective as it provides local release of growth factors and other regenerative proteins secreted by PBSCs thereby improving erectile function. Accordingly, in some embodiments, the therapeutic compositions of the present disclosure comprise PBSCs in addition to the PRP or the growth factor concentrate, along with the thermoresponsive polymer.

In some embodiments, concentration of the PBSCs or the endothelial progenitor cells within the therapeutic composition of the present disclosure ranges from about 10% to 50% (throughout this disclosure, if the concentration of PBSCs is expressed in terms of percentages, it refer to the volume of PBSC solution added to the composition—e.g., 40% PBSCs means 4 ml of PBSC solution is added to make 10 ml of the composition). It is important to note that the compositions of the present disclosure comprise of PRP or GFC, which are derived from whole blood of a subject. Accordingly, as is well known and understood by a person skilled in the art, the internal composition of the whole blood, including the number of cells, proteins, active agents, growth factors etc. varies from subject to subject. Therefore, the PRP or the GFC so prepared varies accordingly, and so do the additional elements, including the PBSCs, and thus arises a need for a range of concentrations within which the compositions of the present disclosure can be prepared and applied. Accordingly, within the ambit of the present disclosure, the concentration of the PBSCs can be any of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%.

In order for the PBSCs or the endothelial progenitor cells to be included in the compositions of the present disclosure, Bone-Marrow Derived Stem Cell (BMSC) mobilization is stimulated by administration of Granulocyte-Colony Stimulating Factor (G-CSF). Administration of GCSF mobilizes BMSCs into peripheral blood to provide PBSCs. Accordingly, in a nonlimiting embodiment, prior to the withdrawal of blood for preparation of the compositions of the present disclosure, the subject is administered with Granulocyte-Colony Stimulating Factor (G-CSF).

In some embodiments, administration of G-CSF enhances the concentration of WBCs in the blood by about 5 to 10-fold, when compared to whole blood analysed without stimulation by G-CSF. As PBSCs play a key role in the process of SC-mediated tissue repair, employing PBSCs in a tissue regenerative composition like the ones of the present disclosure constitutes a therapeutic approach. In view of said rationale, in some embodiments of the present disclosure, a portion of the withdrawn blood is employed to isolate PBSCs, which are then included as part of the compositions of the present disclosure.

In exemplary embodiments, said isolated PBSCs are added to the platelet derived growth factor concentrate for therapeutic applications. The aspect of isolation of PBSCs and their combination with the platelet derived growth factor concentrate of the present disclosure is performed by methods generally known in the art or as further elaborated on in further sections of the present disclosure.

Thus, the present disclosure provides compositions that comprise thermosensitive polymer; conventional PRP or PRP prepared by the present disclosure or the GFC obtained from either of the two PRPs; and peripheral blood stem cells (PBSCs).

In an exemplary embodiment, PBSCs isolated from 10 ml of whole blood sample drawn from a patient pre-treated with GCSF are resuspended in 1 ml of platelet poor plasma to provide 1 ml of PBSC solution which can be employed in preparing the compositions of the present disclosure.

### Thermoresponsive Polymers

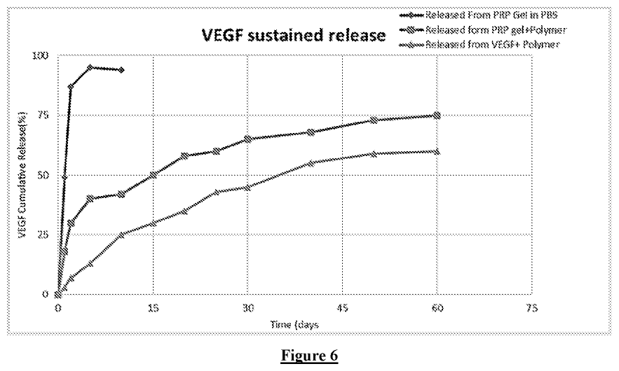

Accordingly, while PRP or GFC forms are the active component of the compositions, it is the thermosensitive polymer that enhances the therapeutic effect by ensuring that the composition is retained by the body at the site of administration for a longer period of time. Since the polymer is thermosensitive in nature, one of the most important properties that it showcases is the conversion of its physical form from liquid to gel, when in contact with physiological temperature such as about 27° C. to 37° C. Thus, in some embodiments, while it is viscous but in the form of an injectable liquid at room temperature, it transitions to a temporary self-forming polymeric plug at body temperature. For example, the thermoresponsive polymer exists in a liquid form at a temperature ranging from about −20° C. to 27° C., and in a gel form at a temperature ranging from about 27.1° C. to 60° C. Because the material undergoes a temperature-induced phase change with no alteration in the product's chemical composition, it works well to enhance the overall impact of the composition. The use of thermoresponsive polymers in the present disclosure therefore allows for sustained and targeted effect of the therapeutic composition of the present disclosure and prevents leakage from the site of administration or dilution by other bodily fluids. Moreover, due to the presence of the thermoresponsive polymer, the composition releases growth factors and/or cells at a slow and sustained rate (FIG. 6).

In some embodiments, the thermoresponsive polymer employed to prepare the compositions of the present disclosure is a synthesized biocompatible polymer, which have no biological contaminants. An example of such a polymer is N-isopropylacrylamide (NIPAM) based polymer, for instance poly(Nisopropylacrylamide-co-n-butyl methacrylate)-poly(NIPAAm-co-BMA). The present disclosure therefore provides for compositions that comprise a NIPAM based polymer; conventional PRP or PRP prepared by the present disclosure or the GFC obtained from either of the two PRPs; optionally along with peripheral blood stem cells (PBSCs), and one or more additional therapeutic agent.

In some embodiments, a thermoresponsive polymer employed to prepare the compositions of the present disclosure includes copolymers composed of thermoresponsive polymer blocks and hydrophilic polymer blocks and is characterized by its temperature-dependent dynamic viscoelastic properties. The thermoresponsive polymer blocks are hydrophilic at temperatures below the sol-gel transition temperature and are hydrophobic at temperatures above the sol-gel transition temperature. The hydrophobic interaction results in formation of a homogenous three-dimensional polymer network in water. In some embodiments, the thermoresponsive polymer block which are part of such copolymers is a NIPAM based polymer. An example of such thermoresponsive polymer blocks is poly(Nisopropylacrylamide-co-n-butyl methacrylate) poly(NIPAAm-co-BMA), which are combined with hydrophilic polymer blocks, including polyethylene glycol (PEG) or poly(lactic-co-glycolic acid), PLGA. The present disclosure therefore provides for compositions that comprise a copolymer of poly(Nisopropylacrylamide-co-n-butyl methacrylate) poly(NIPAAm-co-BMA) and polyethylene glycol (PEG); conventional PRP or PRP prepared by the present disclosure or the GFC obtained from either of the two PRPs; optionally along with peripheral blood stem cells (PBSCs), and one or more additional therapeutic agent. As alternatives to PEG, the thermoresponsive polymers can also comprise poly(D,L-lactide-co-glycolide) (PLGA), poly(lactic acid) (PLA), poly(glutamic acid) (PGA), poly(caprolactone) (PCL), N-(2-hydroxypropyl)-methacrylate (HPMA) copolymers, and poly(amino acids).

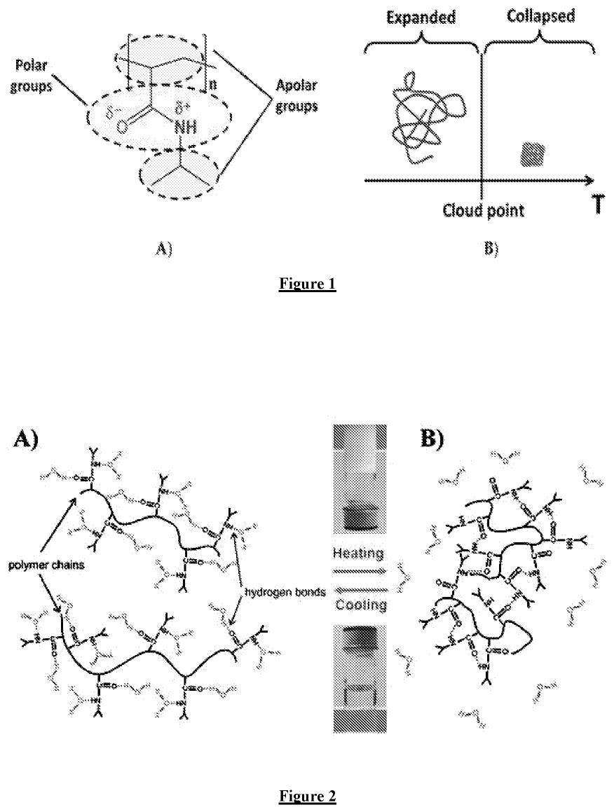

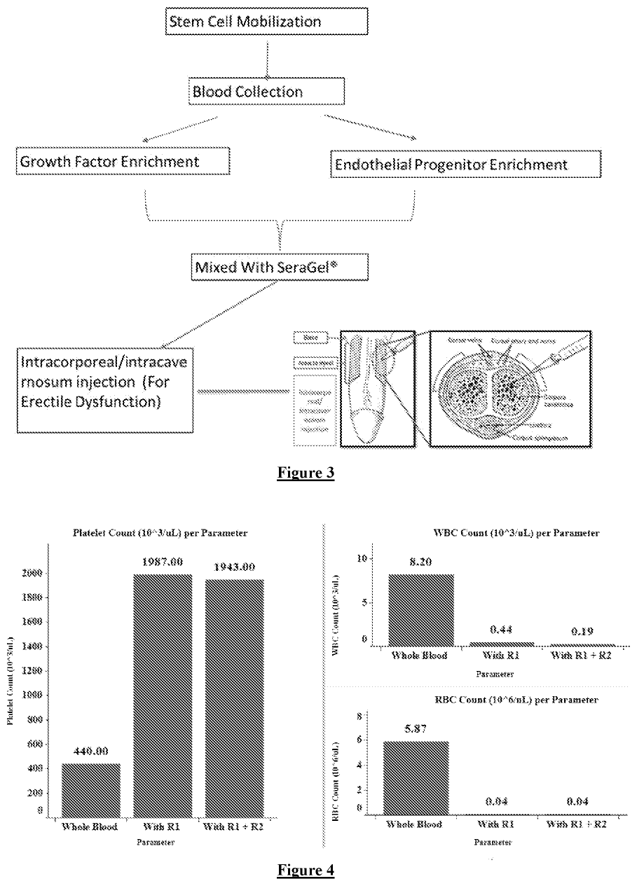

In some embodiments, chemical formula (A) and representation of volume phase transition (B) between coil (left) and globular (right) hydrogel conformations of a NIPAM based polymer is provided in FIG. 1. Further, representation of (A) the swollen PNIPAAm hydro-sol in aqueous solution below critical temperature (Tc) of 32° C. and (B) the shrunken dehydrated PNIPAAm hydrogel above critical temperature (Tc) of 32° C. is provided in FIG. 2.

In some embodiments, the thermoresponsive polymer employed to prepare the compositions of the present disclosure include amphiphilic block copolymers, or ABA triblock copolymers including poloxamers, such as poloxamer 407. These polymers are biocompatible, highly water-soluble and polymorphic materials, and thus ideal for us in thermo sensitive biological applications. While they dissolve conveniently in blood, they are also excreted easily in urine.

In some embodiments, the amphiphilic copolymers include those with hydrophilic block hydrophobic block polymers. An example of such an amphiphilic polymer is a copolymer of poly(ethylene oxide) (PEO) and poly(propylene oxide) (PPO). A commercially available example of such a polymer is Pluronic®.

In some embodiments, a thermoresponsive polymer employed to prepare the compositions of the present disclosure includes any polymer known to a person skilled in the art that possesses thermoresponsive properties. The present disclosure accordingly also contemplates all thermoresponsive polymers that are known in the art, commercially available and/or those approved for medical/therapeutic applications by the U.S. Food and Drug Administration (FDA).

The concentration at which the thermoresponsive polymer may be present within the composition can vary over a range depending on the final constituents of the composition, including PRP, GFC, PBSCs and/or additional therapeutic agents. Similarly, the concentration of the PRP and the GFC within the composition also varies over a specified range.

Thus, in some embodiments, concentration of the thermoresponsive polymer within the therapeutic composition of the present disclosure ranges from about 1% to 50%, including values and ranges therebetween. Accordingly, in the therapeutic compositions, the concentration of the thermoresponsive polymer can range from about 1 to 50%, about 5 to 50%, about 10 to 50%, about 15 to 45%, about 20 to 40%, including values and ranges therebetween. In some embodiments, the concentration of the thermoresponsive polymer can be any of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%.

Accordingly, the PRP or the GFC and the thermoresponsive polymer are present in the compositions of the present disclosure at a ratio ranging from about 90:10 to 10:90, e.g. 900 μl of PRP or GFC and 100 μl of the thermoresponsive polymer and the like. In an exemplary embodiment, a platelet pellet obtained from 10 ml of whole blood is resuspended in 1 ml of platelet-poor plasma to obtain PRP which is further processed as described herein to obtain about 900 μl GFC which is then mixed with 100 μl of the thermoresponsive polymer. Thus, the ratio of GFC and the thermoresponsive polymer in this exemplary embodiment is 90:10.

In embodiments where the composition comprises the PRP or the GFC, PBSCs, and the thermoresponsive polymer; the individual component can be present at a ratio of about 45:45:10 to 5:5:90.

Since the ratio of the PRP or GFC and the thermoresponsive polymer varies from composition to composition depending on the initial constituents of PRP or GFC, and the final application, the present disclosure contemplates all such compositions that satisfy the concentration and ratio requirements set out above. It is important to note that the compositions of the present disclosure comprise of PRP or GFC, which are derived from whole blood of a subject. Accordingly, as is well known and understood by a person skilled in the art, the internal composition of the whole blood, including the number of cells, proteins, active agents, growth factors etc. varies from subject to subject. Therefore, the PRP or the GFC so prepared varies accordingly, and thus arises a need for a range of concentrations within which the compositions of the present disclosure can be prepared and applied.

The present disclosure therefore provides for compositions that comprise a thermoresponsive polymer at a concentration ranging from about 10% to 50%; conventional PRP or PRP prepared by the present disclosure or the GFC obtained from either of the two PRPs at a concentration ranging from about 10% to 90%; optionally along with peripheral blood stem cells (PBSCs) at a concentration ranging from about 10% to 50%, and one or more additional therapeutic agents at a concentration ranging from about 20% to 30%. For example, a composition herein can comprise a thermoresponsive polymer at a concentration of about 20%; conventional PRP or PRP prepared by the present disclosure or the GFC obtained from either of the two PRPs at a concentration of about 30%; along with peripheral blood stem cells (PBSCs) or the endothelial progenitor cells at a concentration of about 50%.

### Additional Therapeutic Agents

In some embodiments of the present disclosure, the compositions herein also comprise one or more additional therapeutic agent selected from a group comprising hormone, growth factor, protein, cells, cell secretome, and drug, or any combination thereof.

In some embodiments, the compositions can comprise additional therapeutic agents selected from the group consisting of: phosphodiesterase V Inhibitors, stem cells (all types from all sources), cells/stem cells secretome, α-1 adrenergic blocker, alprostadil, and a combination thereof. Exemplary phosphodiesterase V Inhibitors that can be added to the compositions include, but are not limited to, sildenafil, vardenafil, tadalafil, and avanafil.

In some embodiments, the compositions are fortified with one or more desired growth factors. For example, patients with ED may naturally have low levels of platelet-derived growth factors. In such cases, compositions of the present disclosure are fortified by exogenously adding growth factor to provide a concentration level that is about 4 to 10 times the physiological levels. Accordingly, within the ambit of the present disclosure, the concentration of the growth factor in the composition can be any of 4-fold, 5-fold, 6-fold, 7-fold, 8-fold, 9-fold or 10-fold of the physiological levels. In some embodiments, compositions of the present disclosure can comprise growth factors selected from the group consisting of: Vascular Endothelial Growth Factor (VEGF), Nerve Growth Factor (NGF), Fibroblast Growth Factor (FGF), Hepatocyte Growth Factor (HGF), Insulin-like growth factor I (IGF-I), Epithelial Growth Factor (EGF), Platelet Derived Growth Factor (PDGF), Stem cell growth factor (SGF), and a combination thereof.

The stem cells that can be included in the composition include adult or embryonic stem cells and from varied sources including those from bone marrow, adipose tissue, blood, umbilical cord and embryo. Further, any drug that is a therapeutic agent known to a person skilled in the art for the treatment of ED, and which can be employed without any compatibility challenges with the compositions of the present disclosure, are also contemplated within the ambit of the present disclosure.

The compositions of the present disclosure can include any combination of these additional agents.

In some embodiments, when the additional therapeutic agent is a hormone, protein, cell, cell secretome, or drug, or any combination thereof, they are present in the composition at a concentration ranging from about 10% to 50%. Accordingly, within the ambit of the present disclosure, the concentration of the additional therapeutic agent in the composition can be any of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%, including values and ranges therebetween.

Thus, the present disclosure provides compositions that comprise thermosensitive polymer; conventional PRP or PRP prepared by the present disclosure or the GFC obtained from either of the two PRPs; peripheral blood stem cells (PBSCs), and one or more additional therapeutic agent. While the present disclosure provides for compositions as captured in this or the previous embodiments, it is important to note that the present disclosure equally contemplates all other possible permutations-combinations that may be possible from the present disclosure, as long as the compositions at minimum comprise conventional PRP or PRP prepared by the present disclosure or GFC obtained from either of the two PRPs; and thermosensitive polymer. Thus, all compositions that comprise both peripheral blood stem cells (PBSCs), and one or more additional therapeutic agent, or comprise only PBSCs without any additional therapeutic agent or comprise only one or more additional therapeutic without any PBSCs are within the ambit of the present disclosure.

### Methods of Preparation

Now in order for the composition of the present disclosure to be manufactured, the present disclosure also provides methods for preparing the therapeutic compositions; methods for preparing conventional PRP or PRP of the present disclosure or the GFC obtained from either of the two PRPs; and methods for peripheral blood stem cells (PBSCs).

In some embodiments, a method for preparing a therapeutic composition comprises mixing the PRP or the growth factor concentrate derived therefrom with the thermoresponsive polymer, optionally along with the PBSCs and additional therapeutic agents, to obtain the composition. It is to be noted that while preparing the compositions of the present disclosure, the thermoresponsive polymer is the last component added to the composition just prior to administration of the composition. That is all components including GFC and optional components like PBSCs and additional therapeutic agents are mixed and the thermoresponsive polymer is added in the end just prior to administration

In some embodiments, the mixing of the components to prepare the composition of the present disclosure is carried out by adding the PRP or GFC in a concentration ranging from about 10% to 90% directly to the thermoresponsive polymer under sterile environment. This thermoresponsive polymer is prepared separately in a liquid selected from water or saline, such as PBS, prior to its mixing with the PRP or the GFC. In some embodiments, the concentration of the thermoresponsive polymer must remain between about 10% to 50% in the final therapeutic composition of the present disclosure.

In some embodiments, depending on the end application of the therapeutic compositions of the present disclosure, the thermoresponsive polymer employed to prepare the composition is in the form of a powder, which is subjected to mixing with water or saline, including PBS, to form a liquid. This liquid is subsequently mixed with the PRP or the GFC to obtain the composition of the present disclosure. However, in alternative embodiments, the thermoresponsive polymer may remain in the form of a powder and mixed directly with the PRP or the GFC to obtain the composition of the present disclosure. In any case, the concentrations of the thermoresponsive polymer within the compositions herein remain within the range provided in the disclosure herein.

In some embodiments, a method for preparing a polymer solution as mentioned above comprises steps of: a) combining an amount of the thermoresponsive polymer or a combination of two polymers (such as NIPAM and PEG) with an amount of a suitable aqueous solvent fortified with growth factors, wherein the amount of polymer(s) is sufficient to form a solution having up to about 10% to 50% w/w of polymer(s); b) stirring the mixture at a sufficiently medium speed at about or below 10° C. at for a first period of time; and c) rocking the mixture for a second period of time thereby forming a solution.

Post contacting of the thermoresponsive polymer with the PRP or GFC, the mixture is cooled in refrigerator or over ice at a temperature ranging from about 2° C. to 10° C. for about 15 minutes. The tube is periodically shaken to help mixing of the contents. Upon dissolving, the mixture is allowed to settle for elimination of air bubbles, post which the mixture, or the composition, is ready for therapeutic administration.

As mentioned, once the thermoresponsive polymer is prepared in the solution form or is obtained in the powder form, it is combined with the PRP or the GFC for preparing the compositions of the present disclosure. Accordingly, the present disclosure also provides for use of the thermoresponsive polymer for preparing the therapeutic composition of the present disclosure.

Thus, in some embodiments, the present disclosure provides for use of the thermoresponsive polymer for preparing a medicament for improving fertility. More particularly, the present disclosure provides for use of the thermoresponsive polymer for preparing a medicament which is the therapeutic composition of the present disclosure for improving fertility in men by treating ED.

In some embodiments, the present disclosure provides for use of the thermoresponsive polymer for preparing a therapeutic compositions for treating infertility caused by ED, wherein the polymer is mixed along with a platelet rich plasma (PRP) or a growth factor concentrate derived therefrom. Of course, in case the compositions of the present disclosure comprise PBSCs and/or additional therapeutic agent(s), the said components also become part of such compositions.

In some embodiments, the medicament prepared by using the thermoresponsive polymer improves fertility by treating ED.

Since the compositions herein also contemplate inclusion of PBSCs or the endothelial progenitor cells and/or one or more additional therapeutic agents, the present disclosure also provides for methods for said inclusion(s) accordingly. The additional therapeutic agents are added to the compositions prior to adding the thermoresponsive polymer

In some embodiments, the PBSCs are added to the compositions of the present disclosure comprising the thermoresponsive polymer and PRP or GFC just prior to administration of the said composition to a subject suffering from ED. In some embodiments of the present disclosure, once the whole blood is collected for the preparation of the PRP or the GFC, a fraction of the blood is kept aside for the preparation of endothelial progenitor cells or PBSCs.

As mentioned previously, since the subject is administered G-CSF one to three days prior to the administration of the composition, the Bone-Marrow Derived Stem Cells (BMSCs) are mobilized leading to circulation of the PBSCs in the blood.

On the day of the administration, the said blood is withdrawn and subjected to enrichment of the PBSCs as described herein. In some embodiments, the PBSCs are prepared in a solution form by the following buffy coat protocol comprising steps of:
a. incubating whole blood collected in an anti-coagulant container with a red blood cell (RBC) aggregating agent selected from the group consisting of: heparin, collagen, a calcium salt, hyaluronic acid, polygeline, thrombin, gelatin, EDTA, sodium citrate, starch, and a combination thereof;
b. subjecting the whole blood to centrifugation at a speed of about 1500 rpm for about 15 minutes high speed;
c. removing top layer containing platelet-poor plasma and transferring middle buffy-coat layer containing PBSCs to another sterile tube;
a) subjecting the buffy coat layer to centrifugation at about 2000 rpm for about 10 minutes or filtration to separate PBSCs to obtain a solution comprising the PBSCs.

Once the solution comprising the PBSCs is prepared, it is mixed with the composition of the thermoresponsive polymer and PRP or GFC at a concentration ranging from about 10% to 50%.

As the compositions herein comprise the PRP of the present disclosure or the GFC obtained therefrom, in order for the said composition to be manufactured, the present disclosure also provides a method for preparing the PRP of the present disclosure. Accordingly, the present disclosure also relates to a method for preparing a PRP, wherein the PRP comprises a platelet count that is about 10 to 20-fold greater than starting whole blood sample, or a RBC count that is about 60 to 90-fold lower than starting whole blood sample, and/or a WBC count that is about 10 to 99-fold lower than starting whole blood sample. The method broadly comprises treating a whole blood sample with one or more RBC aggregating agents, spinning the blood to sediment RBCs and WBCs, spinning the supernatant to sediment platelets, and resuspending the platelets in platelet-poor plasma to provide the PRP.

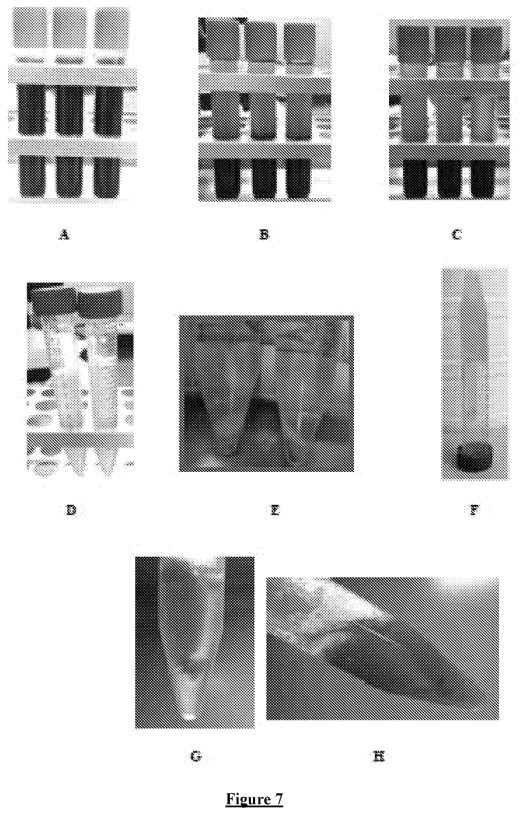

In one embodiment, the method for preparing PRP comprises: (a) incubating a whole blood sample collected in an anti-coagulant container with RBC aggregating agent(s); (b) subjecting the whole blood sample incubated with the RBC aggregating agent to a first centrifugation step to obtain a supernatant containing platelets; (c) subjecting the supernatant to a second centrifugation step to obtain a platelet pellet and platelet-poor plasma (PPP); and (d) resuspending the platelet pellet in PPP to obtain the PRP. FIG. 7, panels A-E show the representative steps for obtaining PRP.

The above described order of steps is not binding on the method of the present disclosure and does not restrict the order in which the steps must be performed. The steps may be performed in any order that is logically feasible, and known to a person skilled in the art.

In some embodiments, the RBC aggregating agent is selected from a group comprising heparin, collagen, a calcium salt, hyaluronic acid, polygeline, thrombin, gelatin, EDTA, sodium citrate, starch, and any combination thereof. In an exemplary embodiment, the RBC aggregating agent is a combination of heparin, collagen, and a calcium salt. In another exemplary embodiment, the RBC aggregating agent is a combination of hyaluronic acid, polygeline, thrombin. In another exemplary embodiment, the RBC aggregating agent is a combination of polygeline, thrombin, and gelatin. In another exemplary embodiment, the RBC aggregating agent is a combination of thrombin, gelatin, and sodium citrate. In another exemplary embodiment, the RBC aggregating agent is a combination of heparin, polygeline, and starch. In another exemplary embodiment, a RBC aggregating agent is a combination of polygeline, gelatin, and starch. In some embodiments, the RBC activating agent is suspended in a physiologically acceptable buffer. In some embodiments, the RBC activating agent is added to the whole blood at a concentration of about 0.2% to 10%, for example, about 0.2, 0.4, 0.6, 0.8, 1.0, 2, 3, 4, 5, 6, 7, 8, 9, or 10% by volume of the whole blood sample. The stock solution of the RBC aggregating agents has a concentration from about 10% to 100%. The whole blood sample is incubated with the RBC activating agent for about 5 to 45 minutes at an ambient temperature. The ambient temperature for incubation ranges from about 4° C. to 37° C., about 10° C. to about 20° C., about 20° C. to 30° C., or about 20° C. to about 25° C. The time of incubation ranges from 5 to 45 minutes, including values and ranges therebetween, such as about 15 minutes, about 20 minutes, about 25 minutes, about 30 minutes, about 35 minutes, about 40 minutes, about 45 minutes, about 15 to 45 minutes, about 30 to 45 minutes, about 10 to 40 minutes, or about 20 to 40 minutes. During the incubation, RBCs aggregate and start settling down.

After incubation with the RBC aggregating agent, the whole blood sample is centrifuged (first centrifugation) at a low speed such as about 300-1000 rpm for about 2-10 minutes. In some embodiments, the first centrifugation step is carried out at about 300 to 1000 rpm, about 350 to 950 rpm, about 350 to 800 rpm, about 400 to 900 rpm, about 450 to 950 rpm, about 400 to 800 rpm, about 500 to 1000 rpm, about 500 to 900 rpm, about 500 to 850 rpm, about 500 to 800 rpm, about 550 to 750 rpm, about 550 to 700 rpm, about 550 to 800 rpm, about 600 to 800 rpm, about 650 to 800 rpm, or about 650 to 750 rpm, including values and ranges therebetween. Time for the first centrifugation step ranges from about 2 to 10 minutes, about 2 to 8 minutes, about 2 to 6 minutes, about 2 to 5 minutes, about 2 to 4 minutes, about 2 to 3 minutes, about 3 to 9 minutes, about 3 to 8 minutes, about 3 to 5 minutes, about 3 to 4 minutes, about 4 to 8 minutes, about 5 to 10 minutes, including values and ranges therebetween. The first centrifugation step can be carried out at any of the speed values for any of the time periods described herein. In the first centrifugation step, RBCs and WBCs sediment and platelets remain in the supernatant. Treatment with RBC aggregating agents prior to the first centrifugation ensures efficient removal of RBCs from the Whole Blood by way of sedimentation.

After the first centrifugation step, the supernatant containing platelets is further centrifuged (second centrifugation step) to sediment platelets. The second centrifugation step is carried out at about 900 to 4000 rpm for about 5-15 minutes. In some embodiments, the second centrifugation step is carried out at about 900 to 3500 rpm, about 1000 to 3000 rpm, about 1200 to 3500 rpm, about 1200 to 3200 rpm, about 1400 to 3500 rpm, about 1400 to 3200 rpm, about 1500 to 3500 rpm, about 1500 to 3200 rpm, about 1500 to 3000 rpm, about 1800 to 3500 rpm, about 1800 to 3200 rpm, about 1800 to 3000 rpm, about 2000 to 3000 rpm, about 2200 to 3200 rpm, about 2500 to about 3200 rpm, about 2500 to 3000 rpm, about 2800 to 3200 rpm, about 2900 to 3100 rpm, including values and ranges therebetween for about 5 to 15 minutes, about 5 to 12 minutes, about 5 to 10 minutes, about 6 to 12 minutes, about 6 to 10 minutes, about 8 to 15 minutes, about 8 to 12 minutes, about 10 to 15 minutes, about 10 to 12 minutes, or about 12 to 15 minutes, including values and ranges therebetween. After the second centrifugation step, platelets form a pellet leaving platelet-poor plasma (PPP) as supernatant. PPP is aspirated and a desired volume of PPP is used to resuspend the platelet pellet to provide platelet-rich plasma. In some embodiments, platelet pellets obtained from about 30 to 60 ml of starting whole blood sample are resuspended in about 3 ml to 6 ml of PPP to provide PRP.

In some embodiments, a method for preparing PRP comprises: (a) incubating a whole blood sample collected in an anti-coagulant container with RBC aggregating agent(s) selected from a group comprising heparin, collagen, a calcium salt, hyaluronic acid, polygeline, thrombin, gelatin, EDTA, sodium citrate, starch, and any combination thereof, wherein the incubation is carried out at a temperature of about 20-25° C.; (b) subjecting the whole blood sample incubated with the RBC aggregating agent to a first centrifugation step to obtain a supernatant containing platelets, wherein the first centrifugation is carried out at about 300-1000 rpm for about 2-10 minutes; (c) subjecting the supernatant to a second centrifugation step to obtain a platelet pellet and platelet-poor plasma (PPP), wherein the second centrifugation is carried out at about 900-4000 rpm for about 5-15 minutes; and (d) resuspending the platelet pellet in PPP to obtain the PRP. Said method for preparing the PRP described herein provides about 10 to 20-fold enrichment of platelets compared to starting whole blood sample, or about 60 to 90-fold reduction in the RBC count compared to starting whole blood sample, and/or about 10 to 99-fold reduction in WBCs, including values and ranges therebetween, compared to starting whole blood sample from same subject.

As the compositions herein, in some embodiments, comprise the GFC instead of the PRP, in order for the composition to be manufactured, the present disclosure also provides a method for preparing the GFC from the conventional PRP or the PRP of the present disclosure. Accordingly, the present disclosure also relates to a method for preparing a growth factor concentrate (GFC) obtained from the PRP prepared according to the methods described herein. That is, in some embodiments, the platelet-derived growth factor concentrate of the present disclosure is prepared from a PRP, wherein the PRP has a platelet count that is about 10 to 20-fold greater than starting whole blood sample, or a RBC count that is about 60 to 90-fold lower than starting whole blood sample, and/or a WBC count that is about 10 to 99-fold lower than starting whole blood sample.

While the GFC of the present disclosure is prepared from the PRP of the present disclosure, the method for preparing which is described herein, it will be understood by a person skilled in the art that similar steps can be applied to conventional PRP for obtaining GFC therefrom. To prepare the GFC, platelets present in the PRP are activated by subjecting the PRP to one or more platelet-activating treatments.

The GFC of the present disclosure is prepared from the PRP of the present disclosure. The methods for preparing the PRP of the present disclosure are described herein. To prepare the GFC, platelets present in the PRP are activated by subjecting the PRP to one or more platelet-activating treatments.

The platelet-activating treatment is selected from a group comprising treatment with platelet activation buffer and free-thaw cycles or a combination thereof.

In some embodiments, the platelet activation buffer comprises platelet activating agent selected from a group comprising collagen, calcium salt, hyaluronic acid, thrombin, and any combination thereof. In exemplary embodiments, the platelet-activating treatment comprises a combination of treatment with platelet activation buffer and one or more freeze-thaw cycles. In some embodiments, the PRP is treated with platelet activation buffer and said treated PRP is subsequently subjected to one or more freeze-thaw cycles. In some embodiments, the Ca salt is calcium chloride or calcium gluconate or other clinically acceptable salts of calcium.

In some embodiments, the platelet activating agents such as collagen, a calcium salt, hyaluronic acid, thrombin, or a combination thereof are provided in a physiologically suitable buffer. In some embodiments, the platelet activating treatment comprises incubating the PRP, for about 15-45 minutes, with a buffer comprising collagen, a calcium salt, and hyaluronic acid. In some embodiments, the platelet activating treatment comprises incubating PRP, for about 15-45 minutes, with a buffer comprising collagen, hyaluronic acid, and thrombin. In some embodiments, the platelet activating treatment comprises incubating PRP, for about 15-45 minutes, with a buffer comprising a calcium salt, hyaluronic acid, and thrombin. In some embodiments, the platelet activating treatment comprises incubating PRP, for about 15-45 minutes, with a buffer comprising a calcium salt and hyaluronic acid followed by or along with or preceding with subjecting the PRP to freeze-thaw cycles. In some embodiments, the platelet activating treatment comprises incubating PRP, for about 15-45 minutes, with a buffer comprising collagen and hyaluronic acid followed by or along with or preceding with subjecting the PRP to freeze-thaw cycles. In some embodiments, the platelet activating treatment comprises incubating PRP, for about 15-45 minutes, with a buffer comprising thrombin and hyaluronic acid followed by or along with or preceding with subjecting the PRP to freeze-thaw cycles. In some embodiments, the platelet activating treatment comprises incubating PRP, for about 15-45 minutes, with a buffer comprising a calcium salt and thrombin followed by or along with or preceding with subjecting the PRP to freeze-thaw cycles. In some embodiments, about 10% to 30% by volume of a buffer containing platelet-activating agents is added to PRP. For example, about 100 microliter of the buffer containing platelet-activating agents is added to 1 ml of PRP.

In some embodiments, the PRP incubated with a buffer containing platelet-activating agents is subjected to 2-7 freeze-thaw cycles. A freeze-thaw cycle comprises freezing the PRP incubated with one or more platelet-activating agents to about 4° C., −20° C., or −80° C., and thawing the frozen PRP at a temperature of about 20° C. to 37° C. or about 25° C. to 37° C. The PRP upon treatment with a platelet-activating treatment forms a gel-like consistency (FIG. 7, panel F). The gel upon standing separates spontaneously from liquid supernatant (FIG. 7, panels G and H). The supernatant contains the GFC.

In some embodiments, the method for preparing GFC comprises: (a) incubating a whole blood sample collected in an anti-coagulant container with RBC aggregating agent(s); (b) subjecting the whole blood sample incubated with the RBC aggregating agent to a first centrifugation step to obtain a supernatant containing platelets; (c) subjecting the supernatant to a second centrifugation step to obtain a platelet pellet and platelet-poor plasma (PPP); and (d) resuspending the platelet pellet in PPP to obtain the PRP; (e) subjecting the PRP to platelet-activating treatment; and (f) collecting supernatant containing the growth factor concentrate.

In some embodiments, the method for preparing GFC comprises (a) incubating a whole blood sample collected in an anti-coagulant container with RBC aggregating agent(s) selected from a group comprising heparin, collagen, a calcium salt, hyaluronic acid, polygeline, thrombin, gelatin, EDTA, sodium citrate, starch, and any combination thereof, wherein the incubation is carried out at a temperature of about 20-25° C.; (b) subjecting the whole blood sample incubated with the RBC aggregating agent to a first centrifugation step to obtain a supernatant containing platelets, wherein the first centrifugation is carried out at about 300-1000 rpm for about 2-10 minutes; (c) subjecting the supernatant to a second centrifugation step to obtain a platelet pellet and platelet-poor plasma (PPP), wherein the second centrifugation is carried out at about 1200-3500 rpm for about 5-15 minutes; and (d) resuspending the platelet pellet in PPP to obtain the PRP (e) activating platelets in the PRP by subjecting the PRP to a platelet-activating treatment selected from a group comprising treatment with platelet activation buffer and free-thaw cycles or a combination thereof, wherein the platelet activation buffer comprises platelet activating agent selected from a group comprising collagen, a calcium salt, hyaluronic acid, thrombin, and any combination thereof; and (f) collecting supernatant containing the growth factor concentrate.

The above described order of steps is not binding on the method of the present disclosure and does not restrict the order in which the steps must be performed. The steps may be performed in any order that is logically feasible. The possibility of supplementing the methods of the present disclosure with steps/modifications routinely practiced in the art in relation to preparation of PRP and platelet derived growth factor compositions is envisaged by the present disclosure.

### Kits

In order to facilitate preparation of the PRP or the GFC of the present disclosure, and subsequently the compositions herein, the present disclosure also provides a kit.

Thus, the present disclosure provides a kit for preparing the therapeutic compositions of the present disclosure, wherein the kit as comprises:
a) a RBC activating agent selected from a group comprising: heparin, collagen, a calcium salt, hyaluronic acid, polygeline, thrombin, gelatin, EDTA, sodium citrate, starch, and a combination thereof;
b) a thermoresponsive polymer; and
c) an instruction manual.

In some embodiments, the kit of the present disclosure further comprises GCSF. In some embodiments, the kit of the present disclosure further comprises a platelet activating agent selected from a group comprising: collagen, a calcium salt, hyaluronic acid, and thrombin, or a combination thereof. The kit also comprises a blood collection container comprising an anticoagulant.

In some embodiments, the kit of the present disclosure further comprises one or more additional therapeutic agents described herein.

As is clear, the kit of the present disclosure is used for preparing the therapeutic compositions herein. In other words, the kit of the present disclosure allows for:
a) processing of whole blood for preparation of PRP of the present disclosure;
b) processing of whole blood for preparation of GFC from the PRP of the present disclosure;
c) processing of conventional PRP for preparation of GFC of the present disclosure;
d) preparing of the therapeutic compositions of the present disclosure comprising PRP and thermosensitive polymer; and/or
e) preparing of the therapeutic compositions of the present disclosure comprising GFC and thermosensitive polymer.

Since the kit comprises the RBC activating agent, in the embodiments where PBSCs are included in the compositions, the kit also facilitates preparation of PBSCs. Accordingly, the kit of the present disclosure also allows for:
a) preparing of the therapeutic compositions of the present disclosure comprising PRP and thermosensitive polymer, and PBSCs; and
b) preparing of the therapeutic compositions of the present disclosure comprising GFC and thermosensitive polymer, and PBSCs.

Further, since the kit comprises one or more additional therapeutic agent, in some embodiments, the kit also facilitates preparation of the compositions of the present disclosure having said additional therapeutic agent.

In some embodiments, the kit comprises an instruction manual having steps for: processing of the whole blood for processing of whole blood for preparation of PRP of the present disclosure; processing of whole blood for preparation of GFC from the PRP of the present disclosure; processing of conventional PRP for preparation of GFC of the present disclosure; preparing of the therapeutic compositions of the present disclosure comprising PRP and thermosensitive polymer; and/or preparing of the therapeutic compositions of the present disclosure comprising GFC and thermosensitive polymer. The instructional manual may additionally comprise steps for processing of PBSCs and/or inclusion on additional therapeutic agent during preparation of any of the said compositions.

It is to be understood by a person skilled in the art that the embodiments relating to the use of the kit on possibilities of processing the blood, and/or preparing the compositions herein are only exemplary in nature, and all possible permutations-combinations that are possible within the ambit of the present disclosure are equally applicable to the use of the kit, as long as the kit is able to facilitate the said processing or preparation.

### Methods of Use/Treatment

The present disclosure also provides use of the therapeutic compositions of the present disclosure in treating male fertility caused due to ED.

In some embodiments, provided herein is a method for treating erectile dysfunction in a subject in need thereof comprising, administering to the subject any of the therapeutic compositions described herein. In some embodiments, the subject is treated with GCSF prior to administration of the therapeutic composition.

In some embodiments, the therapeutic composition is administered once every month for two months. In some embodiments, booster doses of the compositions are administered after 6 months, 9 months, 12 months, and/or 15 months from the first dose.

In some embodiments, methods for treating erectile dysfunction of the present disclosure improve the International Index of Erectile Function (IIEF) score from less than 10 to about 13 to 20, to about 13 to 18, to about 14 to 19, to about 15 to 20, to about 13 to 25, to about 13 to 23, to about 14 to 24, to about 15 to 25, including values and ranges therebetween.

In some embodiments, therapeutic compositions are administered into the penis of the subject via intracavernosal or intracorporeal injection.

In some embodiments, the therapeutic composition is administered to the penis in an amount ranging from about 5 ml to about 10 ml. Accordingly, the therapeutic composition is administered to the penis in an amount of about 5 ml, 6 ml, 7 ml, 8 ml, 9 ml, or 10 ml.

As the compositions of the present disclosure comprise of PRP or the growth factor concentrate, the underlying growth factors present therein help in the treatment due to its well-known regenerative potential. When PRP or PRP derived GFC is administered to the penis, the growth factors improve erectile functionality by increasing blood flow to the penis, increasing girth of the penis, and increasing stamina, sensation, and pleasure.

As the present disclosure contemplates inclusion of PBSCs in the compositions herein, in some embodiments, the subject is administered G-CSF for a period of one to three days prior to the administration of the composition of the present disclosure. Accordingly, in some embodiments, on the day of the treatment by administration of the composition, the following process is performed:
a) withdrawal of whole blood, e.g., about 60-80 ml, followed by or along with or preceding with optional segregation into two fractions—one for preparing the composition and another for preparing the solution containing the PBSCs. Alternatively, two separate fractions can be withdrawn from the subject for the two activities;
b) employing the first fraction to prepare the composition of the present disclosure, and the second fraction for preparing the concentrated solution containing the PBSCs;
c) mixing of the prepared composition with the concentrated solution of the PBSCs to arrive at the final composition for administering to the subject in need of treatment for ED.

The detailed steps involved in preparation of the composition as mentioned in the preceding embodiment, along with preparation of the solution containing PBSCs are as per the methods provided in the present disclosure.

In alternative embodiments, in case no PBSCs are included in the final composition, the step of G-CSF administration and preparation of the solution containing PBSCs is eliminated.

Since the compositions of the present disclosure comprise a thermoresponsive polymer, it is to be noted that while the composition will be in a liquid form during the preparation and administration, owing to its temperature sensitive nature, the composition comprising the thermoresponsive polymer will convert into a gel form upon contact with physiological temperature. This will allow the composition to be retained by the penis, and avoid dilution of the delivered material and result in sustained localised delivery of the composition.

While the instant disclosure is susceptible to various modifications and alternative forms, specific aspects thereof have been shown by way of examples and drawings and are described in detail below. However, it should be understood that it is not intended to limit the invention to the particular forms disclosed, but on the contrary, the intention is to cover all modifications, equivalents, and alternatives falling within the spirit and the scope of the invention as defined by the appended claims.

### EXAMPLES

### Example 1: Preparation of Platelet-Rich Plasma (PRP)

A 30 ml of venous blood was drawn from a patient and 10 ml each was aliquoted into acid citrate dextrose (ACD-A) solution gel tube/K2 EDTA tube. The samples were incubated for 45 minutes with a buffer comprising polygeline, gelatin, and starch as RBC aggregating agents. After incubation, samples were centrifuged at 600 rpm for 2 minutes. Supernatant containing platelets was collected and again centrifuged at 3000 rpm for 12 minutes. After this centrifugation, platelets sedimented as a pellet and the supernatant contained platelet-poor plasma (PPP). The platelet pellet was resuspended in 3 ml of PPP to obtain PRP. FIG. 7, panels A-E, show various stages during the PRP preparation.

The number of platelets, RBCs, and WBCs in the PRP were counted. Table 2 below shows the cell count obtained by the above-described method (PRP of the present disclosure) and comparative cell count obtained by conventional PRP methods. The cell count values for conventional PRP methods are based on the values disclosed in “Principles and Methods of Preparation of Platelet-Rich Plasma: A Review and Author's Perspective”, J Cutan Aesthet Surg. 2014 October-December; 7(4): 189-197.doi: 10.4103/0974-2077.

TABLE 2
Cell count of conventional PRP and the PRP of the present disclosure

| | | | Fold | | |
|---|---|---|---|---|---|
| | | | increase | | |
| | | Platelets | over | Total WBC | RBC |
| | | Count | whole | Count | Count |
| | Parameters | 10\hat{ }3/ul | blood | 10\hat{ }3/ul | 10\hat{ }6/ul |
| 1 | Whole Blood (Minimum | 150 | — | 4.5 | 4.7 |
| | Normal Value) | | | | |
| 2 | Conventional PRP Protocol | 1096 | 7.4 | 12.6 | 8.9 |
| | (Single Spin/Buffy Coat Method) | | | | |
| | Conventional PRP Protocol | 1577 | 10.5 | 11.3 | 1.1 |
| | (Double Spin/PRP method) | | | | |
| 3 | PRP Method of the | 2023 | 13.4 (1.8 | 0.19 (23.6 | 0.06 (78.33 |
| | present disclosure | | fold over | fold | fold |
| | | | single | reduction | reduction |
| | | | spin/1.3 | over whole | over whole |
| | | | fold over | blood/66.3 | blood/148.3 |
| | | | double | fold | fold |
| | | | spin) | reduction | reduction |
| | | | | over single | over single |
| | | | | spin/59.47 | spin/18.33 |
| | | | | fold | fold |
| | | | | reduction | reduction |
| | | | | over double | over double |
| | | | | spin) | spin) |

### Example 2: Preparation of Platelet-Derived Growth Factor Concentrate (GFC)

PRP was prepared as described in Example 1. 300 μl of a platelet activation buffer comprising calcium chloride and thrombin was mixed with the PRP and the mixture was incubated for 45 minutes. After incubation, the mixture was subjected to three freeze-thaw cycles with freezing at 4° C. and thawing at 37° C. The supernatant containing the GFC was collected and aliquoted into cryovials, which can be used for administration right away or can be preserved for future use. FIG. 7, panels F-H, show the stages during the GFC preparation.

ELISA assays were performed to determine levels of growth factors present in the freshly-prepared GFC and the levels upon storage at 20° C. or −10° C. Table 3 below shows the levels in the freshly-prepared GFC and the levels upon storage at 20° C. for a duration of 3, 6, 9, and 12 hours.

TABLE 3
Freshly-prepared and upon storage at 20° C.

| | pg/ml | pg/ml | pg/ml | ng/ml | ng/ml | ng/ml |
|---|---|---|---|---|---|---|
| Duration | VEGF | EGF | bFGF | IGF-1 | PDGF-BB | TGF-b1 |
| Fresh | 914 ± 400 | 183 ± 50 | 50.2 ± 24.0 | 102.7 ± 26.5 | 53.2 ± 32.3 | 294 ± 45.2 |
| 1 h | 901 ± 390 | 190.2 ± 34.2 | 54 ± 22.7 | 108.5 ± 28.4 | 60.2 ± 22.4 | 310.2 ± 34.2 |
| 3 h | 850.2 ± 381.2 | 178 ± 43.2 | 47 ± 21.4 | 98.7 ± 26.5 | 57 ± 21.4 | 280 ± 48.2 |
| 6 h | 839.1 ± 390.6 | 160 ± 46.2 | 45 ± 23.5 | 93.7 ± 25.5 | 43 ± 27.5 | 290 ± 46.2 |
| 9 h | 222.4 ± 45.3 | 65 ± 22.4 | 19 ± 10.5 | 22.3 ± 18.2 | 21 ± 11.5 | 135 ± 23.4 |
| 12 h | 112 ± 45.3 | 46 ± 20.4 | 18 ± 23.5 | 24.4 ± 17.5 | 14 ± 13.5 | 60.2 ± 22.4 |

Table 4 below shows the levels in the freshly-prepared GFC and the levels upon storage at −10° C. for a duration of 1 week, 4 weeks, 8 weeks, 12 weeks and 24 weeks.

TABLE 4
Freshly-prepared and upon storage at −10° C.

| | VEGF | EGF | bFGF | IGF-1 | PDGF-BB | TGF-b1 |
|---|---|---|---|---|---|---|
| Duration | pg/ml | pg/ml | pg/ml | ng/ml | ng/ml | ng/ml |
| Fresh | 914 | 183 | 50.2 | 102.7 | 53.2 | 294 |
| 1 w | 890 | 190 | 58 | 110 | 62 | 260 |
| 4 w | 850.2 | 210 | 51 | 97 | 56 | 280 |
| 8 w | 839.1 | 170 | 47 | 93.7 | 43 | 290 |
| 12 w | 890 | 200 | 50 | 82 | 49 | 240 |
| 24 w | 860 | 160 | 46 | 96 | 51 | 270 |

### Example 3: Preparation of Peripheral Blood Stem Cells (PBSCs)

A 10 ml of venous blood was drawn from a patient into an acid citrate dextrose (ACD-A) solution gel tube/K2 EDTA tube. The sample was incubated for 45 minutes with a buffer comprising polygeline, gelatin, and starch as RBC aggregating agents. After incubation, samples were centrifuged at 1500 rpm for 10 minutes. Upon centrifugation, RBCs, WBCs, and platelets were separated as follows: the bottom layer contained RBCs, the middle layer contained platelets and WBCs (buffy coat layer) and the top layer was platelet-poor plasma. The top layer (PPP) was removed and the middle buffy coat layer was transferred to another sterile tube. The tube was centrifuge at 2000 rpm for 12 minutes to separate WBCs. Alternatively, leucocyte filtration filter can be used to separate WBCs. The table 5 below shows the WBC, RBC, and platelet count of the PBSC solution obtained using this method. The numbers in parenthesis in the last column indicate fold increase over whole blood.

TABLE 5
Cell count of PBSCs

| | Whole blood | Buffy |
|---|---|---|
| Parameters | (Range) | coat/PBSCs |
| WBC (×10\hat{ }3/ul) | 1.44-30.75 | 5 (5×) |
| RBC (×10\hat{ }6/ul) | 1.66-5.96 | 1.0 |
| PLT (×10\hat{ }3/ul) | 150-450 | 690 (>4×) |

### Example 4: Analysis of the Effect of RBC Aggregators on the PRP Profile

Example 1 was repeated with the following variations—
A) Employment of a single RBC aggregator—gelatin
B) Employment of a combination of 2 RBC aggregators—gelatin+starch
C) Employment of no RBC aggregator
D)-F) No RBC aggregators

Experiments A-F were designed to have gradually increasing centrifugation speed and time. G was a control experiment.

Specifics of the above experiments are depicted in Table 6 below.

TABLE 6
Blood Processing for PRP - Protocol Standardization

| Step | Parameter | A | B | C | D | E | F | G |
|---|---|---|---|---|---|---|---|---|
| 1 | RBC | With | With | Whole | | | | |
| | aggregators | RBC1 | RBC1 + 2 | Blood | | | | |
| 2 | Incubation | 15 | 30 | 45 | No | | | |
| | time-minutes | | | | | | | |
| 3 | Centrifugation- | 500 | 600 | 700 | 800 | 900 | 1000 | No |
| | rpm | | | | | | | |
| 4 | Centrifugation- | 2 | 4 | 6 | 8 | 10 | | |
| | time-minutes | | | | | | | |
| 5 | Platelet | Ca Salt- | Thrombin- | Ca + | Freeze-Thaw | Freeze- | | |
| | activation | 45 mins | 45 mins | Thrombin- | (4degree- | Thaw LN2 | | |
| | | | | 45 mins | 37degree/ | 10 mins/ | | |
| | | | | | 10 mins/ | cyclex3 | | |
| | | | | | cyclex3 | | | |

| | | |
|---|---|---|
| 4 | GFC assay- | 9*5 Assays |
| | ELISA | |

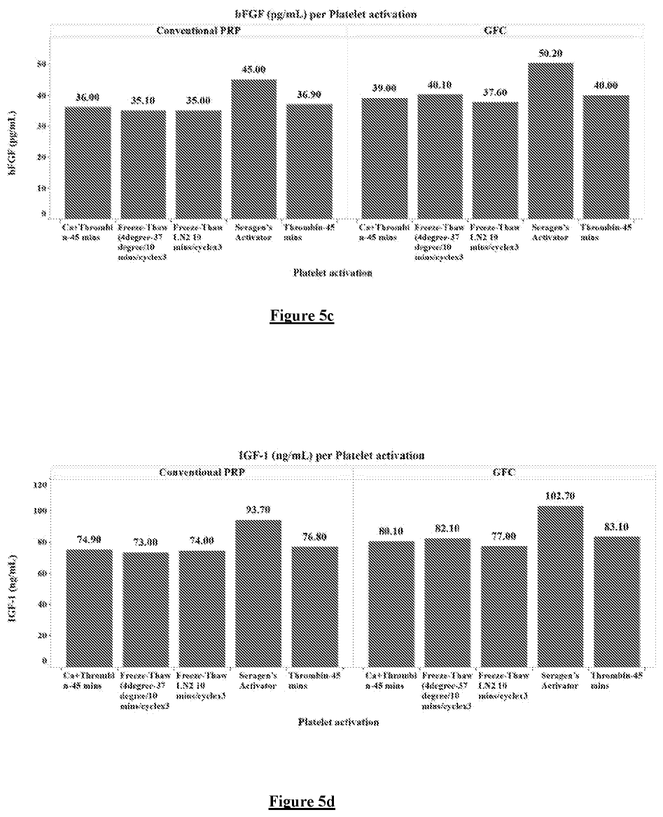

Experiments A and B which employed RBC aggregators were found to yield improved results with respect to settling and separation of RBCs and WBCs through their respective protocols. For reasons of brevity, results from variations of the experiment closest to the protocol of the present disclosure are depicted as graphs in FIG. 4. As can be observed from said figure, the incorporation of RBC aggregators in the PRP/GFC preparation protocol has a significant impact in terms of the improvement in platelet count and reduction in RBC and WBC count. The combination of 2 RBC aggregators was found to further improve the reduction in WBC count in the PRP.

### Example 5: Analysis of the Effect of Different Platelet Activation Protocols

The effect of the choice of platelet activation protocol on the concentration of growth factors in the final GFC was analyzed by performing variations of the experiment in Example 2. Keeping other specifics of the experiment constant, said variations employed treatment of PRP with single platelet activating agent, treatment of PRP with a combination of 2 activating agents, exposure of PRP to freeze-thaw cycles at different temperatures and a combination of treatment of PRP with activation agent and exposure to freeze-thaw cycles. Between freezing and thawing.

Results yielded by said experiments are provided in Table 7 below. The parameter entitled “Freeze-Thaw (4 degree-37 degree/10 mins/cycle×3)” indicates that the samples were frozen and kept as frozen for 10 minutes; the samples were then thawed and kept as thawed for 10 minutes; and these steps were repeated three times. The parameter entitled (Freeze-Thaw LN2 10 mins/cycle×3)” indicates that the freezing was carried out at −196. In the parameter entitled “Activation buffer+Freeze-Thaw cycles”, a Ca salt was included in the activation buffer as a platelet activating agent.

TABLE 7

| | | VEGF | EGF | bFGF | IGF-1 | PDGF-BB | TGF-b1 |
|---|---|---|---|---|---|---|---|
| Platelet activation | | (pg/ml) | (pg/ml) | (pg/ml) | (ng/ml) | (ng/ml) | (ng/ml) |
| PRP of the | Activation buffer - | 740 ± 80 | 148 ± 30 | 40 ± 22 | 83.1 ± 23 | 43 ± 28.9 | 238 ± 35.6 |
| present | Thrombin-45 mins | | | | | | |
| disclosure | Activation buffer - | 712 ± 395 | 142 ± 42 | 39 ± 19 | 80.1 ± 19.2 | 41 ± 21.3 | 229 ± 31.2 |
| | Ca + Thrombin-45 mins | | | | | | |
| | Freeze-Thaw (4degree- | 731 ± 372 | 146 ± 51 | 40.1 ± 15 | 82.1 ± 14.3 | 42.5 ± 18.7 | 235 ± 29 |
| | 37degree/10 mins/cyclex3 | | | | | | |
| | Freeze-Thaw LN2 | 685 ± 437 | 137 ± 40 | 37.6 ± 10 | 77 ± 21.1 | 39.9 ± 19.5 | 220 ± 25 |
| | 10 mins/cyclex3 | | | | | | |
| | Activation buffer + | 914 ± 400 | 183 ± 50 | 50.2 ± 24.0 | 102.7 ± 26.5 | 53.2 ± 32.3 | 294 ± 45.2 |
| | Freeze-Thaw cycles | | | | | | |
| Conventional | Thrombin-45 mins | 687.9 ± 370 | 131 ± 41.3 | 36.9 ± 19.4 | 76.8 ± 24.4 | 35 ± 23.4 | 237.8 ± 41.2 |
| PRP | Ca + Thrombin- | 671.2 ± 362 | 128 ± 43.2 | 36 ± 18.9 | 74.9 ± 19.2 | 34.4 ± 18.3 | 232 ± 38.7 |
| | 45 mins | | | | | | |
| | Freeze-Thaw (4degree- | 654 ± 358 | 124.8 ± 35.2 | 35.1 ± 21.1 | 73 ± 14.4 | 33.5 ± 19.6 | 226.2 ± 39.2 |
| | 37degree/10 mins/cyclex3 | | | | | | |
| | Freeze-Thaw LN2 | 662 ± 379 | 126.4 ± 39.1 | 35.55 ± 17.3 | 74 ± 19.5 | 33.9 ± 16.2 | 229.1 ± 42 |
| | 10 mins/cyclex3 | | | | | | |
| | Activation buffer + | 839.1 ± 390.6 | 160 ± 46.2 | 45 ± 23.5 | 93.7 ± 25.5 | 43 ± 27.5 | 290 ± 46.2 |
| | Freeze-Thaw cycles | | | | | | |

Observations from the above experiments show that a platelet activation protocol employing a combination of treatment of PRP with activation agent and exposure of PRP to freeze-thaw cycles yields GFC with significantly higher growth factor concentration—said effect being observed for both the PRP of the present disclosure as well as conventional PRP.

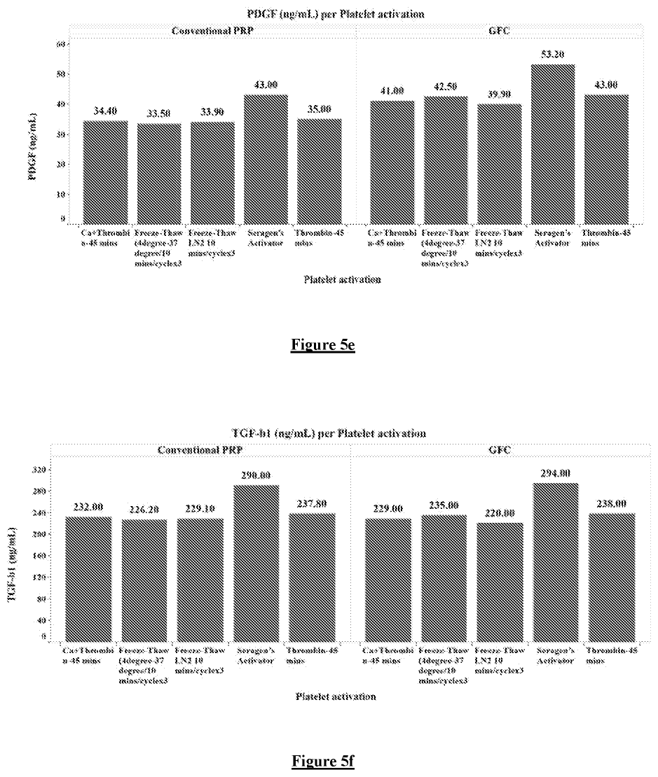

The PRP of the present disclosure, however, provides a notably higher concentration of individual growth factors in the GFC derived therefrom when compared to conventional PRP that is subjected to platelet activation by the same protocol. Thus, a synergy between the PRP preparation protocol and PRP activation protocol in yielding GFC with high growth factor concentration is derivable from the above data. The above results are depicted in FIG. 5.

### Example 6: Preparation of Composition Comprising PRP and Thermoresponsive Polymer

For preparing a composition comprising PRP and thermoresponsive polymer [(NIPAM based polymer—poly(Nisopropylacrylamide-co-n-butyl methacrylate) poly(NIPAAm-co-BMA)], the first step was to obtain the PRP. As described in the present disclosure, the PRP can either be obtained from whole blood by conventionally known methods, or by specific protocol as recited in example 1 above.

In the present example, the objective was to prepare 10 ml of the composition for administration into penis of an ED subject. Accordingly, about 5 ml of the PRP prepared by the exemplified protocol was taken for mixing with 5 ml or 50% (as a final concentration) of the thermoresponsive polymer.

Separately, the thermoresponsive polymer, which was in the form of a powder, was subjected to mixing with water or phosphate-buffered saline to form a solution having a concentration of about 50%. For this, the following steps were performed:
a) the thermoresponsive polymer was dissolved in 50 ml amount of water to obtain a solution having up to about 50% w/w of polymer(s);
b) the solution was stirred at medium speed (30-100 rpm) at about 10° C. at for a first period of time (about 15 minutes); and
c) the mixture was rocked for a second period of time (about 15 minutes) thereby forming a solution.

In an alternate experiment, the thermoresponsive polymer, was directly taken in the form of a powder for mixing with the PRP, without dissolution in water or saline.

Accordingly, two batches of mixtures were prepared. One comprising about 5 ml of the PRP and 5 ml of the solution of the polymer; and the second comprising about 10 ml of the PRP and 0.5 g of the polymer powder (50%). For preparation of these mixtures, the following steps were performed:
a) the thermoresponsive polymer was contacted with the PRP in a sterile tube, and the mixture was cooled in refrigerator at a temperature of about 4° C. for about 15 minutes;
b) the tube was periodically shaken to help mixing of the contents and maintained at the same temperature;
c) once dissolved, the mixture was allowed to settle for elimination of air bubbles.

This mixture comprised of 5 ml of PRP and 5 ml or 50% of the thermoresponsive polymer.

This experiment was subsequently repeated by replacing the NIPAM based polymer with Poloxamer 407 to obtain a composition comprising PRP and Poloxamer 407.

These final compositions were prepared for administration to an ED subject.

### Example 7: Preparation of Composition Comprising GFC and Thermoresponsive Polymer

For preparing a composition comprising GFC and thermoresponsive polymer [(NIPAM based polymer—poly(Nisopropylacrylamide-co-n-butyl methacrylate) poly(NIPAAm-co-BMA)], the first step was to obtain the GFC. As described in the present disclosure, the GFC can either be obtained from conventionally known PRP, or by specific protocol as recited in example 2 above.

In the present example, the objective was to prepare 10 ml of the composition for administration into penis of an ED subject. Accordingly, about 5 ml of the GFC prepared by the exemplified protocol was taken for mixing with 5 ml or 50% (as a final concentration) of the thermoresponsive polymer.

Separately, the thermoresponsive polymer, which was in the form of a powder, was subjected to mixing with water or saline to form a solution having a concentration of about 50%. For this, the following steps were performed:
d) the thermoresponsive polymer was dissolved in 50 ml of water to obtain a solution having up to about 50% w/w of polymer(s);
e) the solution was stirred at medium speed (30-100 rpm) at about 10° C. at for a first period of time (15 minutes); and
f) the mixture was rocked for a second period of time (15 minutes) thereby forming a solution.

Accordingly, two batches of mixtures were prepared. One comprising about 5 ml of the GFC and 5 ml of the solution of the polymer; and the second comprising about 10 ml of the GFC and 0.5 g of the polymer or the powder sufficient for (50%). For preparation of these mixtures, the following steps were performed:
a) the thermoresponsive polymer was contacted with the PRP in a sterile tube, and the mixture was cooled in refrigerator at a temperature of about 8° C. for about 10 minutes;
b) the tube was periodically shaken to help mixing of the contents and maintained at the same temperature;
c) once dissolved, the mixture was allowed to settle for elimination of air bubbles.

This mixture comprised of 5 ml of GFC and 5 ml or 50% of the thermoresponsive polymer.

This experiment was subsequently repeated by replacing the NIPAM based polymer with Poloxamer 407 to obtain a composition comprising GFC and Poloxamer 407.

These final compositions were prepared for administration to an ED subject.

### Example 8: Preparation of Composition Comprising PRP or GFC and Thermoresponsive Polymer Along with PBSCs

For preparing a composition comprising PRP or GFC and thermoresponsive polymer [(NIPAM based polymer—poly(Nisopropylacrylamide-co-n-butyl methacrylate) poly(NIPAAm-co-BMA)], along with PBSCs, the first step was to obtain the PRP or the GFC. As described in the present disclosure, the PRP can either be obtained from conventionally known PRP, or by specific protocol as recited in example 1 above. Similarly, the GFC can either be obtained from conventionally known PRP, or by specific protocol as recited in example 2 above.

In the present example, the objective was to prepare 10 ml of the composition for administration into penis of an ED subject. Accordingly, about 4 ml of the PRP prepared by the exemplified protocol was taken for mixing with 2 ml or 20% (as a final concentration) of the thermoresponsive polymer. In an alternate experiment, about 4 ml of the GFC prepared by the exemplified protocol was taken for mixing with 2 ml or 20% (as a final concentration) of the thermoresponsive polymer.

Separately, four batches of the thermoresponsive polymer were prepared—two in solution form (similar to examples 6 and 7 above) and two directly in the powder form.

Separately, four fractions of 4 ml (40% of the final composition) of the PBSCs were prepared from the whole blood of the subject, as per the buffy coat protocol described in example 3 above.

For preparing the final compositions, four batches of initial mixtures were prepared, that comprised of PRP or GFC and PBSCs for final mixing with the polymer as follows:

PRP and PBSC for mixing with polymer in powder form;

PRP and PBSC for mixing with polymer in solution form;

GFC and PBSC for mixing with polymer in powder form; and

GFC and PBSC for mixing with polymer in solution form.

Each of these batches comprised of about 4 ml of the PRP or GFC respectively and about 4 ml or 40% of the PBSCs. For preparation of these mixtures, simple mixing steps were carried out.

To these 4 batches, the 4 fractions of 2 ml (20%) of the polymer was added, to prepare the final composition for administration to an ED subject. For preparation of these final mixtures, mixing steps similar to those in examples 6 and 7 were followed. The following table 8 provides for the particulars of the composition prepared herein:

TABLE 8

| Particulars | |
|---|---|
| PBSCs (V %) | 50 |
| GFC/PRP (V %) | 30 |
| Polymer (V %) | 20 |
| Final volume (ml) | 1 |

This experiment was subsequently repeated by replacing the NIPAM based polymer with Poloxamer 407 to obtain a composition comprising PRP or GFC, Poloxamer 407 and PBSCs.

### Example 9: Preparation of Composition Comprising PRP or GFC and Thermoresponsive Polymer with Additional Therapeutic Agent

For preparing a composition comprising PRP or GFC and thermoresponsive polymer [(NIPAM based polymer—poly(Nisopropylacrylamide-co-n-butyl methacrylate) poly(NIPAAm-co-BMA) or Poloxamer 407], with or without PBSCs, and with additional therapeutic agent, the overall protocol remained the same as those described in the previous examples. The additional therapeutic agent was added to PRP, GFC, or GFC+PBSCs, and the polymer was added last.

### Example 10: Effect of Thermoresponsive Polymer on Release Profile of the Composition Comprising PRP or Recombinant Growth Factor

This example was designed for assessing the importance of the thermoresponsive polymer in the compositions of the present disclosure. This was carried out by comparing the growth factor release profile from a composition comprising the polymer, and a composition devoid of it. For further analysis on the effect of the polymer, regardless of the underlying active component, a test composition of recombinantly prepared VEGF with the polymer was also prepared.

In this example, composition comprising PRP and thermoresponsive polymer was prepared as per the protocol provided in example 6 above. To compare the effect of the said polymer, a preparation of PRP (as per the protocol of example 1 above) in equal volume of phosphate buffer saline was prepared. The test composition of recombinant VEGF with the polymer, was prepared by a simple 1:1 mixing of the recombinant VEGF with the polymer.

The in vitro growth factor release kinetics was performed in PBS (pH 7.4) at 37° C. for 60 days as reported in FIG. 6. As can be seen, VEGF released from PRP mixed with polymer within the first 2 days (burst effect) was 30±3%, followed by a phase of sustained release with almost 75% of VEGF being released within 60 days (orange/middle graph). Although, the VEGF release was lower for composition of recombinant VEGF mixed with polymer, it still showed good profiling over the full 60 day period (gray/third graph from top). However, in contrast, no release of growth factors was observed for the preparation of PRP in PBS beyond the first 10 days (blue/first graph from top). Accordingly, it is evident that the composition devoid of the polymer lost any ability for sustained effect because of the dilution. However, very clearly, the polymer supports the sustained delivery of growth factors in both the compositions that had it. The growth factor release from the polymer validates the slow release of these proteins for long term availability and therapeutic efficacy.

### Example 11: Treatment of Patients with Erectile Dysfunction with a Composition Comprising Autologous PBSCs and Autologous Platelet-Derived GFC

In this study, 150 patients with chronic organic erectile dysfunction duration of at least 0.5 years were enrolled. The patients has an IIEF (Baseline International Index of Erectile Function) score of <26. Patients were divided into a treatment group (N=100) and a control group (N=50).

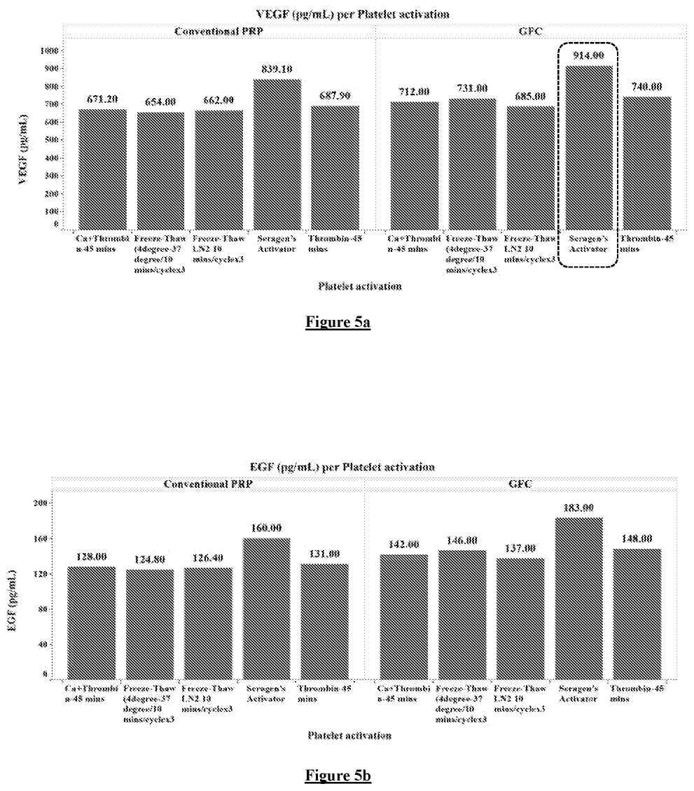

In the treatment group, patients were administered GCSF subcutaneously once a day for 2 days. On the third day, after GCSF administration, 80 ml of venous blood was drawn from the patients into acid citrate dextrose (ACD-A) solution gel tube/K2 EDTA tubes. 60 ml of blood was processed as described in Example 3 to prepare a 6 ml solution comprising autologous PBSCs. 20 ml of blood was processed as described in Example 2 to prepare a 2 ml of autologous platelet-derived growth factor concentrate (GFC). A composition comprising 6 ml of PBSCs and 2 ml of GFC was prepared. A single 8 ml injection of this composition was administered into the corpus cavernosum of the patient's penis (intracavernosal injection) as shown in FIG. 3.

A rubber band placed on the patient during the intracavernosal injection was removed after 30 minutes. Immediate negative pressure pump was initiated with a penis pump. Negative pressure is important, if delayed, the GFC can return to general circulation within a patient's body. Immediate pumping increases the availability of growth factors and cytokines for the cells present throughout the penis. Daily pumping is necessary to produce microtearing within the corpus cavernosum continuing the use of the growth factors for the growth of new muscle tissues and vasculature. It was recommended to the patients to apply ice packs on the penis for few minutes followed by hot water bags. This helped in ensuring liquidation of gel thereby releasing components for few minutes followed by gel formation when heat is applied.

The control group received the standard of care treatment.

Observation was assessed after 4 weeks of treatment and 3 months, with IIEF (International Index of Erectile Function) score, penile triplex, and a greater number of morning erections to interpret the improvement post GFC/PBSCS intervention. The average pre treatment IIEF score was 8.6 and post GFC/PBSCS score was 16.9 with the conditional improvement of 90-95% & their Erectile functionality includes increased blood flow to the penis, substantial increase in girth, an increase in stamina, sensation, and pleasure were determined to prove the overall therapeutic potential of ErectSERA™. 100% of patients reported no side effects except injection induced manageable pain and discomfort. Overall, 70 out of 100 (70%) men recovered their erectile function, and could implement sexual intercourse after 6 months and the effect sustained 12 months after GFC/stem cell treatment.

Using the generally-accepted IIEF questionnaire to measure erectile function, the whole group of 100 patients reported that their score had increased from 8 before the stem cell transplantation surgery, to 14 after 6 months. However, in the “only PRP treatment” group of men who recovered sexual function, the IIEF score increased from 9 to 14 (the average in men with ‘normal’ sexual function is around 25). The satisfaction score in the GFC/PBSCs group was higher and effect duration was also longer in this group. This is enough to enable some of the men to have a spontaneous erection sufficient for penetrative sex, others achieved this with the help of medication.

Table 9 below is the breakup of results by age and condition.

TABLE 9

| Parameters | GFC/PBSCs | PRP |
|---|---|---|
| No of patients treated | 100 | 100 |
| Follow up | 75 Patients | 80 Patients |
| International index | 8.6 | 9.2 |
| of erectile function | | |
| (IIEF-5) Score- | | |
| Pre treatment-avg | | |
| International index | 19 | 14.8 |
| of erectile function | | |
| (IIEF-5) Score- | | |
| Post treatment-Avg | | |
| Improvement in | 70 (70%) | 62 (62%) |
| Erection Hardness | | |

The results indicate that a single intracavernosal injection of a composition comprising freshly isolated autologous PBSCs and freshly-prepared autologous GFC statistically significantly improved erectile function in the study group.

### Example 12: Animal Studies with Compositions of the Present Disclosure

Male Sprague-Dawley rats were randomly divided into three groups: Group I underwent sham operation, while the remaining two groups underwent bilateral CN crush. Crush injury groups were treated at the time of injury with an application of PRP, GFC and GFC mixed with polymer (PNIPAAm-PEG) or phosphate buffered saline only injection in the corpus cavernosum, respectively. Four weeks later, erectile function (EF) was assessed by CN electrosimulation, and CNs as well as penile tissue were collected for histology.

Four weeks after surgery, in the vehicle only group (PBS), the functional evaluation showed a lower mean maximal ICP than that in the sham group (P<0.05). While PRP and GFC treatments resulted in EF recovery, GFC with polymer treatments resulted in significant recovery of EF, as compared with the vehicle-only group. Histologically, the GFC with polymer-treated group had a significant preservation of myelinated axons of CNs compared with the vehicle-only and PRP alone group and reduced the apoptotic index. The mRNA expression of TGF-b1 in the corpus cavernosum tissue was significantly decreased in the PRP, GFC and GFC with polymer group compared with the vehicle-only group. It was clear demonstration of GFC with/without polymer injection in the corpus cavernosum increased the number of myelinated axons and facilitated recovery of EF in the bilateral CN injury rat model.

The results are summarized in Table 10 below.

TABLE 10

| | No of animals | | |
|---|---|---|---|
| | 30 | 30 | 30 |
| | Control/ | Control/ | Control/PBS/GFC + |
| | PBS/ | PBS/ | thermoresponsive |
| | PRP | GFC | polymer |
| Intracavernous Pressure | 120/43/70 | 120/43/87 | 120/43/102 |
| Luxol Fast Blue | +++/−/++ | +++/−/++ | +++/−/+++ |
| Staining of CN and | | | |
| Penile Dorsal Nerve | | | |
| Area of Myelinated | 67/19/43 | 67/19/49 | 67/19/56 |
| axon/cavernous nerve % | | | |
| Apoptptic Cell No/ | 2.8/31/14 | 2.8/31/9 | 2.8/31/7 |
| 160000 um\hat{ }2 | | | |

### Example 13: Preparation of Kit of the Present Disclosure

A kit was prepared in accordance with the requirements of the present disclosure. The kit so prepared comprises of the following components:
a) G-CSF;
b) a RBC activating agent selected from a group comprising: heparin, collagen, a calcium salt, hyaluronic acid, polygeline, thrombin, gelatin, EDTA, sodium citrate, starch, and a combination thereof;
c) a thermoresponsive polymer; and
d) an instruction manual.

The kit was prepared in a manner so that it can be used for the following:
a) processing of whole blood for preparation of PRP of the present disclosure as per example 1 above;
b) processing of whole blood for preparation of GFC from the PRP of the present disclosure as per example 2 above;
c) processing of conventional PRP for preparation of GFC of the present disclosure as per example 2 above;
d) preparing of the therapeutic compositions of the present disclosure comprising PRP and thermosensitive polymer as per example 6 above;
e) preparing of the therapeutic compositions of the present disclosure comprising GFC and thermosensitive polymer as per example 7 above;
f) preparing of the therapeutic compositions of the present disclosure comprising PRP and thermosensitive polymer, and PBSCs as per example 8 above; and/or
g) preparing of the therapeutic compositions of the present disclosure comprising GFC and thermosensitive polymer, and PBSCs as per example 8 above.

In addition to the above 4 components, separate kits were also prepared to comprise one platelet activating agent selected from a group comprising collagen, a calcium salt, hyaluronic acid, and thrombin.

In all these kits, a blood collection container comprising an anti-coagulant was also provided.

All the kits so prepared herein additionally comprise an instruction manual each having steps for: processing of the whole blood for processing of whole blood for preparation of PRP of the present disclosure; processing of whole blood for preparation of GFC from the PRP of the present disclosure; processing of conventional PRP for preparation of GFC of the present disclosure; preparing of the therapeutic compositions of the present disclosure comprising PRP and thermosensitive polymer; and preparing of the therapeutic compositions of the present disclosure comprising GFC and thermosensitive polymer. The instructional manual also comprises steps for processing of PBSCs and inclusion on additional therapeutic agent during preparation of any of the said compositions.

Additional embodiments and features of the present disclosure will be apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, Band C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring 25 back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to anyone of claims 1 to 3, it follows that the combination of the subject-matter of claims 4 and 1, of claims 4, 2 and 1, of claims 4, 3 and 1, as well as of claims 4,3,2 and 1 is clearly and unambiguously disclosed.

The above considerations apply mutatis mutandis to all attached claims. To give a few examples, the combination of claims 6, 5, 4(b), 3 and 2 is clearly and unambiguously envisaged in view of the claim structure. The same applies for the combinations of claims 6, 35 5, 4(a), 3 and 2, and, to give a few further examples which are not limiting, the combination of claim 4(a) and 2 and the combination of claim 5, 4(a) and 2.

1. A therapeutic composition comprising a platelet rich plasma (PRP) or a growth factor concentrate derived therefrom and a thermoresponsive polymer.

2. The therapeutic composition of claim 1, wherein the PRP is a conventional PRP; or a PRP having a platelet count that is about 10 to 20-fold greater than starting whole blood sample from same subject, a red blood cell (RBC) count that is about 60 to 90-fold lower than starting whole blood sample from same subject, a white blood cell (WBC) count that is about 10 to 99-fold lower than starting whole blood sample from same subject, or any combination thereof.

3. The therapeutic composition of claim 1, wherein the growth factor concentrate comprises growth factor(s) selected from the group consisting of: VEGF, EGF, bFGF, IGF-1, PDGF-BB, TGF-β1, and a combination thereof.

4. The therapeutic composition of claim 1, wherein concentration of the VEGF ranges from about 500 to 3000 pg/mL, concentration of the EGF ranges from about 100 to 3000 pg/mL, concentration of the bFGF ranges from about 25 to 3000 pg/mL, concentration of the IGF-1 ranges from about 500 to 3000 ng/mL, concentration of the PDGF-BB ranges from about 20 to 3000 ng/mL, and concentration of the TGF-β1 ranges from about 100 to 3000 ng/mL.

5. The therapeutic composition of claim 1, comprising peripheral blood stem cells (PBSCs).

6. The therapeutic composition of claim 1, wherein the PRP or the growth factor concentrate derived therefrom or the PBSCs is autologous or can be derived from umbilical cord blood, bone marrow, fresh/expired platelet concentrates from blood banks, buffy coat from blood banks.

7. The therapeutic composition of claim 1, comprising an additional therapeutic agent selected from the group consisting of: a growth factor, a phosphodiesterase V inhibitor, stem cells, a cell secretome, an α-1 adrenergic blocker, alprostadil, and a combination thereof; wherein the growth factor, if present, is selected from the group consisting of: VEGF, Nerve Growth Factor (NGF), FGF, HGF, IGF-1, EGF, PDGF, stem cell growth factor (SGF), and a combination thereof.

8. The therapeutic composition of claim 1, wherein the thermoresponsive polymer is selected from the group consisting of: a copolymer comprising poly(N-isopropylacrylamide-co-n-butyl methacrylate) and polyethylene glycol; a copolymer comprising poly(N-isopropylacrylamide-co-n-butyl methacrylate) and poly(lactic-co-glycolic acid); a copolymer comprising poly(ethylene oxide) (PEO) and poly(propylene oxide) (PPO); a NIPAM based polymer; amphiphilic block copolymers; ABA triblock copolymers; poloxamer; and a combination thereof; and wherein the thermoresponsive polymer exists in a liquid form at a temperature ranging from about −20° C. to 27° C., and in a gel form at a temperature ranging from about 27.1° C. to 60° C.

9. The therapeutic composition of claim 1, wherein concentration of the thermoresponsive polymer ranges from about 10% to 50%.

10. The therapeutic composition of claim 1, wherein the PRP or the growth factor concentrate and the thermoresponsive polymer are present at a ratio of about 90:10 to 50:50; or wherein the PRP or the growth factor concentrate, the PBSCs, and thermoresponsive polymer are present at a ratio of about 45:45:10 to 5:5:90.

11. A method for preparing the therapeutic composition of claim 1, comprising mixing the PRP or the growth factor concentrate derived therefrom with the thermoresponsive polymer to obtain the composition.

12. The method of claim 11, comprising adding peripheral blood stem cells to the composition.

13. The method of claim 11, wherein the PRP or the growth factor concentrate is mixed with the thermoresponsive polymer at a ratio of about 90:10 to 10:90 or wherein the PRP or the growth factor concentrate, the PBSCs, and thermoresponsive polymer are mixed at a ratio of about 45:45:10 to 5:5:90.

14. The method of claim 11, comprising mixing the composition with an additional therapeutic agent selected from the group consisting of: a growth factor, a phosphodiesterase V inhibitor, stem cells, a cell secretome, an α-1 adrenergic blocker, alprostadil, and a combination thereof; wherein the growth factor, if added, is selected from the group consisting of: VEGF, Nerve Growth Factor (NGF), FGF, HGF, IGF-1, EGF, PDGF, stem cell growth factor (SGF), and a combination thereof.

15. The method of claim 11, wherein the PRP is prepared by a method comprising:
incubating whole blood with a red blood cell (RBC) aggregating agent selected from a group comprising: heparin, collagen, a calcium salt, hyaluronic acid, polygeline, thrombin, gelatin, EDTA, sodium citrate and starch, or any combination thereof;
subjecting the whole blood incubated with the RBC aggregating agent to a first centrifugation to obtain a supernatant containing platelets;
subjecting the supernatant to a second centrifugation to obtain a platelet pellet and platelet-poor plasma (PPP);
resuspending the platelet pellet in PPP to obtain the PRP.

16. The method of claim 11, wherein the growth factor concentrate derived from the PRP is prepared by a method comprising:
activating platelets in the PRP obtained by the method of claim 15, by treating the PRP with a platelet-activating treatment selected from a group comprising: collagen, a calcium salt, hyaluronic acid, thrombin, and freeze-thaw cycles, or any combination thereof; and
collecting supernatant containing the growth factor concentrate.

17. The method of claim 15, wherein the whole blood is incubated with the RBC aggregating agent for about 5-45 minutes; and wherein the RBC aggregating agent is added at a concentration of about 0.1 to 10% by volume of the whole blood.

18. The method of claim 15, wherein the first centrifugation is carried out at a speed of about 300 rpm to 1000 rpm for about 1-5 minutes; and wherein the second centrifugation is carried out at a speed of about 1200 rpm to 2500 rpm for about 10-15 minutes.

19. The method of claim 16, wherein the platelet-activating treatment comprises treating the PRP with a platelet activating agent selected from collagen, a calcium salt, hyaluronic acid, thrombin, and a combination thereof, followed by or along with or preceding with or along with or preceding with one or more freeze-thaw cycles.

20. The method of claim 12, wherein the PBSCs are prepared by a method comprising:
incubating whole blood collected in an anti-coagulant container with a red blood cell (RBC) aggregating agent selected from the group consisting of: heparin, collagen, a calcium salt, hyaluronic acid, polygeline, thrombin, gelatin, EDTA, sodium citrate, starch, and a combination thereof;
subjecting the whole blood to centrifugation at about 1500 rpm for about 15 minutes;
removing top layer containing platelet-poor plasma and transferring middle buffy-coat layer containing PBSCs to another sterile tube;
subjecting the buffy coat layer to centrifugation at about 2000 rpm for about 10 minutes or filtration to separate PBSCs to obtain a solution comprising the PBSCs.

21-34. (canceled)
